(19) United States
(12) Patent Application Publication
(10) Pub. No.: US 2026/0226450 A1
(43) Pub. Date: Aug. 6, 2026

(54) GENOME ENGINEERED YEAST WITH HIGH GLUTATHIONE CONTENT

(71) Applicant: Lesaffre et Cie SA, Paris (FR)

(72) Inventors: Garri Arzumanyan, Irvine, CA (US); Massimo Merighi, Quincy, MA (US); Justin Smith, Pacifica, CA (US); Bob St. Onge, San Francisco, CA (US)

(73) Assignee: Lesaffre et Cie SA, Paris (FR)

(21) Appl. No.: 19/432,548

(22) Filed: Dec. 24, 2025

Related U.S. Application Data
(60) Provisional application No. 63/738,926, filed on Dec. 26, 2024.

(51) Int. Cl. C12N 9/00; C12N 1/18; C12R 1/865

(52) U.S. Cl. CPC C12N 9/93; C12N 1/18; C12Y 603/02002; C12Y 603/02003; C12N 2830/34; C12R 2001/865

(57) ABSTRACT

Provided are gamma glutamylcysteine synthetase variants and microbial cells comprising such variants and/or expressing heterologous gamma-glutamylcysteine synthetase-glutathione synthetases, for producing increased amounts of the antioxidant peptide glutathione (GSH). Methods and compositions provided herein may be useful in baking and winemaking applications and in production of nutraceuticals, pharmaceuticals, cosmetics, plant protectants and/or animal feeds.

### RELATED APPLICATIONS

This application claims the benefit under 35 U.S.C. § 119(e) of United States Provisional Application No. 63/738,926, filed Dec. 26, 2024, entitled GENOME ENGINEERED YEAST WITH HIGH GLUTATHIONE CONTENT, the entire disclosure of which is hereby incorporated by reference in its entirety.

### REFERENCE TO AN ELECTRONIC SEQUENCE LISTING

The contents of the electronic sequence listing (R090170001US01-SEQ-OMJ.xml; Size: 14,976 bytes; and Date of Creation: Dec. 19, 2025) are herein incorporated by reference in their entirety.

### FIELD

The present disclosure relates to yeast that produces high levels of glutathione (GSH).

### BACKGROUND

Glutathione (GSH) is a biologically abundant thiol tripeptide that is present across multiple kingdoms of life, including animals, plants, fungi, and bacteria. GSH possesses antioxidant properties that confer important cellular fitness benefits. Accordingly, the peptide has diverse applications in, e.g., food, pharmaceutical, nutraceutical, and cosmetic industries.

### SUMMARY

Aspects of the present disclosure provide gamma glutamylcysteine synthetases that are characterized by reduced binding of GSH and thus reduced feedback inhibition by GSH, thereby achieving high-titer production of GSH in microbes, such as yeast. Further provided are microbial cells (e.g., yeast cells) that contain heterologous genes encoding bifunctional glutathione synthesis enzymes of bacterial origin (GCS-GS) that are naturally resistant to GSH feedback inhibition.

Aspects of the disclosure relate to gamma glutamylcysteine synthetases comprising an amino acid sequence that is at least 70%, 75%, 80%, 85%, 90%, or 95% identical to the sequence of SEQ ID NO: 1, wherein the amino acid sequence of the gamma glutamylcysteine synthetase comprises an amino acid substitution relative to the sequence of SEQ ID NO: 1 at one or more amino acid positions listed in Table 1 or Table 2.

In some embodiments, the amino acid sequence of the gamma glutamylcysteine synthetase comprises an amino acid substitution relative to the sequence of SEQ ID NO: 1 at two or more amino acid positions listed in Table 1 or Table 2.

In some embodiments, the amino acid sequence of the gamma glutamylcysteine synthetase comprises an amino acid substitution at one or more positions corresponding to positions K220, L238, C264, N277, V312, or G319 of SEQ ID NO: 1. In some embodiments, the amino acid sequence of the gamma glutamylcysteine synthetase further comprises an amino acid substitution at a position corresponding to position C266 of SEQ ID NO: 1.

In some embodiments, the amino acid sequence of the gamma glutamylcysteine synthetase comprises an amino acid substitution at two or more positions corresponding to positions K220, L238, C264, C266, N277, V312, or G319 of SEQ ID NO: 1. In some embodiments, the amino acid sequence of the gamma glutamylcysteine synthetase comprises one or more of the following amino acid substitutions relative to the sequence of SEQ ID NO: 1: K220E, L238H, C264L, C266A, N277D, V312I, or G319R.

In some embodiments, the amino acid sequence of the gamma glutamylcysteine synthetase comprises the following amino acid substitutions relative to the sequence of SEQ ID NO: 1: C266A, N277D, and V312I; K220E, L238H, and C266A; K220E, L238H, C266A, and V312I; or L238H, C264L, C266A, and G319R.

In some embodiments, the gamma glutamylcysteine synthetase is resistant to feedback inhibition by glutathione (GSH). In some embodiments, the gamma glutamylcysteine synthetase maintains enzymatic activity of a control gamma glutamylcysteine synthetase of SEQ ID NO: 1.

Further aspects of the disclosure relate to microbial cells, such as yeast cells, comprising gamma glutamylcysteine synthetases described herein. In some embodiments, the yeast cell is a S. cerevisiae cell. In some embodiments, the yeast cell is diploid. In some embodiments, the yeast cell is triploid. In some embodiments, the yeast cell is tetraploid. In some embodiments, the yeast cell exhibits increased GSH production relative to a control yeast cell.

In some embodiments, the yeast cell further comprises a heterologous glutathione biosynthesis bifunctional protein. In some embodiments, the heterologous glutathione biosynthesis bifunctional protein is a bacterial glutathione biosynthesis bifunctional protein. In some embodiments, the bacterial glutathione biosynthesis bifunctional protein is a S. thermophilus or S. agalactiae glutathione biosynthesis bifunctional protein. In some embodiments, the heterologous glutathione biosynthesis bifunctional protein is expressed in the yeast cell under the control of a TDH3 promoter, a Rp118b promoter, or a RNR2 promoter. In some embodiments, the yeast cell exhibits increased GSH production relative to a control yeast cell. In some embodiments, the glutathione biosynthesis bifunctional protein comprises an amino acid sequence that is at least 70%, 75%, 8-%, 85%, 90%, or 95% identical to the sequence of SEQ ID NO: 3 or SEQ ID NO: 5, or a conservatively substituted version thereof.

Further aspects of the disclosure relate to compositions comprising yeast cells described herein. In some embodiments, the composition comprises one or more of: yeast extract, active dry yeast, instant dry yeast, crumbled yeast, cream yeast, or baker's yeast.

Further aspects of the disclosure relate to compositions comprising GSH purified from yeast cells described herein. In some embodiments, the composition is for use in breadmaking. In some embodiments, the composition is baker's dough. In some embodiments, the composition is for use in winemaking. In some embodiments, the composition is for production of a nutraceutical. In some embodiments, the composition is for production of a pharmaceutical. In some embodiments, the composition is for production of a plant protectant.

Further aspects of the disclosure relate to methods for increasing antioxidant content in a product comprising using a yeast cell or composition described herein, wherein the product is a bread or a wine.

Further aspects of the disclosure relate to methods for increasing antioxidant content in a product comprising using a yeast cell or composition described herein, wherein the product is a nutraceutical or a pharmaceutical.

Further aspects of the disclosure relate to methods for increasing antioxidant content in a product comprising using a yeast cell or composition described herein, wherein the product is a plant protectant.

Further aspects of the disclosure relate to yeast cells comprising: (a) a heterologous glutathione biosynthesis bifunctional protein; and (b) a gamma glutamylcysteine synthetase comprising an amino acid sequence that is at least 70%, 75%, 8-%, 85%, 90%, or 95% identical to the sequence of SEQ ID NO: 1, wherein the amino acid sequence of the gamma glutamylcysteine synthetase comprises an amino acid substitution relative to the sequence of SEQ ID NO: 1 at one or more amino acid positions listed in Table 1 or Table 2. In some embodiments, the glutathione biosynthesis bifunctional protein comprises an amino acid sequence that is at least 70%, 75%, 8-%, 85%, 90%, or 95% identical to the sequence of SEQ ID NO: 3 or SEQ ID NO: 5, or a conservatively substituted version thereof. Further aspects of the disclosure relate to methods for increasing antioxidant content of a product comprising using such a yeast cell, wherein the product is a bread, a wine, a nutraceutical, a pharmaceutical, or a plant protectant.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. The drawings are illustrative only and are not required for enablement of the disclosure. For purposes of clarity, not every component may be labeled in every drawing. In the drawings:

FIGS. 1A-1C depict a screen described in Example 1. FIG. 1A provides a schematic of the experimental workflow used to screen a Gsh1 site saturation mutagenesis (SSM) library for ticlatone sensitivity. FIGS. 1B and 1C depict results from the screen. Each data point represents a unique Gsh1 variant strain arranged by position of the amino acid alteration on the x-axis and Ticlatone sensitivity on the y-axis. Data points are colored by variant type in FIG. 1B or by subpool in FIG. 1C.

FIGS. 2A-2B depict heatmaps representing Gsh1 SSM growth profiling results for Cadmium (FIG. 2A) and Ticlatone (FIG. 2B). Gsh1 amino acid position are arranged along the x-axis with the 20 possible amino acids arranged on the y-axis. #=stop codon.

FIG. 3A shows BSO resistance of Gsh1 SSM library members. Each data point represents a different amino acid substitution. Results for strains in which said substitution is coupled to upstream (y-axis) or downstream (x-axis) synonymous changes are compared. Mutations indicated with an “X” conferred BSO resistance in both synonymous genetic backgrounds. FIG. 3B shows a summary (average of upstream and downstream scores) of cadmium, ticlatone and BSO data for amino acid substitutions indicated with an “X” FIG. 3A. Shaded cells indicate sensitivity to Ticlatone, Cadmium, or both (an indication of Gsh1 activity loss). Gsh1 mutations shown with an asterisk represent mutations that confer resistance to BSO while maintaining sufficient activity to confer resistance to Ticlatone and Cadmium.

FIG. 4 shows glutathione levels for strains expressing bacterial GCS-GS enzymes from S. thermophilus (St) or S. agalactiae (Sa) using different strength promoters and in different genetic backgrounds. Intracellular glutathione (y-axis) was quantified in a microtiter plate using the fluorescent probe, Thioltracker. The average of two or more replicates are plotted. Error bars represent the standard deviation.

FIG. 5 shows GSH titers of YR043 derived strains expressing StGCS-GS from different promoters following culture in shake flask. GSH was quantified using HPLC and is represented on the y-axis in grams per 100 g of yeast dry weight.

FIG. 6 shows GSH titers (g/100 g YDW) as measured in the YR043 background via shake-flask and HPLC. GSH1 and StGCS-GS genotypes are indicated below.

### DETAILED DESCRIPTION

Aspects of the disclosure relate to methods and compositions for producing increased amounts of the antioxidant peptide GSH. Provided are variants of Gsh1 that confer resistance to feedback inhibition by GSH but maintain high activity of the enzyme, and yeast cells expressing such variants. Further provided are yeast cells that express heterologous genes encoding bifunctional glutathione synthesis enzymes of bacterial origin (GCS-GS). Both types of genetic changes circumvent feedback inhibition of the biosynthetic pathway by the product GSH, thereby achieving high-titer production of GSH in yeast. Yeast cells described herein and GSH produced by such cells, are useful for a variety of applications, including, for example, breadmaking, production of fermented beverages, production of nutraceuticals and/or pharmaceuticals, production of cosmetics, production of plant protectants, and/or production of animal feed.

### Gamma Glutamylcysteine Synthetase

Aspects of the disclosure relate to gamma glutamylcysteine synthetase enzymes. As used herein, a “gamma glutamylcysteine synthetase,” also called “glutamate-cysteine ligase,” classified in EC 6.3.2.2, refers to an enzyme in the cellular glutathione (GSH) biosynthetic pathway that catalyzes the chemical reaction:

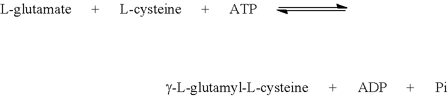

Gamma glutamylcysteine synthetases from any source may be compatible with aspects of this disclosure. Gamma glutamylcysteine synthetases may be naturally occurring or may be synthetic. In some embodiments, a gamma glutamylcysteine synthetase is derived from yeast.

GSH1 is a Saccharomyces cerevisiae (S. cerevisiae) gene that encodes the S. cerevisiae protein Gamma glutamylcysteine synthetase (Gsh1), which produces Glu-Cys dipeptide from the amino acids glutamate and cysteine. Gsh2 encoded by a S. cerevisiae GSH2 gene adds glycine to the Glu-Cys dipeptide to generate the GSH tripeptide. Gsh1 is the rate limiting enzyme in yeast GSH biosynthesis. Gsh1 is feedback inhibited by the end-product GSH, thus limiting levels of GSH produced by yeast fermentation. Biosynthesis of GSH in yeast is regulated by competitive feedback inhibition of Gsh1 by GSH. When bound to Gsh1, GSH occupies the glutamate and presumed cysteine binding site of the enzyme, thereby preventing additional synthesis of GSH. This mechanism limits the amount of GSH which can be produced by yeast fermentation, leading to products with insufficient GSH content and higher production costs.

Yeast strains can comprise multiple alleles of GSH1. In some embodiments, different alleles of GSH1 encode for a Gsh1 protein with the same amino acid sequence, while in other embodiments, different alleles of GSH1 encode for Gsh1 proteins with different amino acid sequences.

In some embodiments, a Gsh1 protein comprises the sequence of SEQ ID NO: 1.

(SEQ ID NO: 1)
MGLLALGTPLQWFESRTYNEHIRDEGIEQLLYIFQAAGKRDNDPLFWGDELEYMVV
DFDDKERNSMLDVCHDKILTELNMEDSSLCEANDVSFHPEYGRYMLEATPASPYLN
YVGSYVEVNMQKRRAIAEYKLSEYARQDSKNNLHVGSRSVPLTLTVFPRMGCPDFI
NIKDPWNHKNAASRSLFLPDEVINRHVRFPNLTASIRTRRGEKVCMNVPMYKDIATP
ETDDSIYDRDWFLPEDKEAKLASKPGFIYMDSMGFGMGCSCLQVTFQAPNINKARY
LYDALVNFAPIMLAFSAAAPAFKGWLADQDVRWNVISGAVDDRTPKERGVAPLLP
KYNKNGFGGIAKDVQDKVLEIPKSRYSSVDLFLGGSKFFNRTYNDTNVPINEKVLGR
LLENDKAPLDYDLAKHFAHLYIRDPVSTFEELLNQDNKTSSNHFENIQSTNWQTLRF
KPPTQQATPDKKDSPGWRVEFRPFEVQLLDFENAAYSVLIYLIVDSILTESDNINAYIH
MSKVWENMKIAHHRDAILFEKFHWKKSFRNDTDVETEDYSISEIFHNPENGIFPQFV
TPILCQKGFVTKDWKELKHSSKHERLYYYLKLISDRASGELPTTAKFFRNFVLQHPD
YKHDSKISKSINYDLLSTCDRLTHLDDSKGELTSFLGAEIAEYVKKNKPSIESKC*.

In non-limiting embodiments, the Gsh1 protein comprising the sequence of SEQ ID NO: 1 may be encoded by a polynucleotide comprising the sequence of SEQ ID NO: 2.

(SEQ ID NO: 2)
ATGGGACTCTTAGCTTTGGGCACGCCTTTGCAGTGGTTTGAGTCTAGGACGTACA
ATGAACACATAAGGGATGAAGGTATCGAGCAGTTGTTGTATATTTTCCAAGCTG
CTGGTAAAAGAGACAATGACCCTCTTTTTTGGGGAGACGAGCTTGAGTACATGG
TTGTAGATTTTGATGATAAGGAGAGAAATTCTATGCTCGACGTTTGCCATGACAA
GATACTCACTGAGCTTAATATGGAGGATTCGTCCCTTTGTGAGGCTAACGATGTG
AGTTTTCACCCTGAGTATGGCCGGTATATGTTAGAGGCAACACCAGCTTCTCCAT
ATTTGAATTACGTGGGTAGTTACGTTGAGGTTAACATGCAAAAAAGACGTGCCA
TTGCAGAATATAAGCTATCTGAATATGCGAGACAAGATAGTAAAAATAACTTGC
ATGTGGGCTCCAGGTCTGTCCCTTTGACGCTGACTGTCTTCCCGAGGATGGGATG
CCCCGACTTTATTAACATTAAGGATCCGTGGAATCATAAAAATGCCGCTTCCAGG
TCTCTGTTTTTACCCGATGAAGTCATTAACAGACATGTCAGGTTTCCTAACTTGA
CAGCATCCATCAGGACCAGGCGTGGTGAAAAAGTTTGCATGAATGTTCCCATGT
ATAAAGATATAGCTACTCCAGAAACGGATGACTCCATCTACGATCGAGATTGGT
TTTTACCAGAAGACAAAGAGGCGAAACTGGCTTCCAAACCGGGTTTCATTTATAT
GGATTCCATGGGTTTTGGCATGGGCTGTTCGTGCTTACAAGTGACCTTTCAGGCA
CCCAATATCAACAAGGCACGTTACCTGTACGATGCATTAGTGAATTTTGCACCTA
TAATGCTAGCCTTCTCTGCCGCTGCGCCTGCTTTTAAAGGTTGGCTAGCCGACCA
AGATGTTCGTTGGAATGTGATATCTGGTGCGGTGGACGACCGTACTCCGAAGGA
AAGAGGTGTTGCGCCATTACTACCCAAATACAACAAGAACGGATTTGGAGGCAT
TGCCAAAGACGTACAAGATAAAGTCCTTGAAATACCAAAGTCAAGATATAGTTC
GGTTGATCTTTTCTTGGGTGGGTCGAAATTTTTCAATAGGACTTATAACGACACA
AATGTACCTATTAATGAAAAAGTATTAGGACGACTACTAGAGAATGATAAGGCG
CCACTGGACTATGATCTTGCTAAACATTTTGCGCATCTCTACATAAGAGATCCAG
TATCTACATTCGAAGAACTGTTGAATCAGGACAACAAAACGTCTTCAAATCACTT
TGAAAACATCCAAAGTACAAATTGGCAGACATTACGTTTTAAACCCCCCACACA
ACAAGCAACCCCGGACAAAAAGGATTCTCCTGGTTGGAGAGTGGAATTCAGACC
ATTTGAAGTGCAACTATTAGATTTTGAGAACGCTGCGTATTCCGTGCTCATATAC
TTGATTGTCGATAGCATTTTGACCTTTTCCGATAATATTAACGCATATATTCATAT
GTCCAAAGTATGGGAAAATATGAAGATAGCCCATCACAGAGATGCTATCCTATT
TGAAAAATTTCATTGGAAAAAATCATTTCGCAACGACACCGATGTGGAAACTGA
AGATTATTCTATAAGCGAGATTTTCCATAATCCAGAGAATGGTATATTTCCTCAA
TTTGTTACGCCAATCCTATGCCAAAAAGGGTTTGTAACCAAAGATTGGAAAGAA
TTAAAGCATTCTTCCAAACACGAGAGACTATACTATTATTTAAAGCTAATTTCTG
ATAGAGCAAGCGGTGAATTGCCAACAACAGCAAAATTCTTTAGAAATTTTGTAC
TACAACATCCAGATTACAAACATGATTCAAAAATTTCAAAGTCGATCAATTATG
ATTTGCTTTCTACGTGTGATAGACTTACCCATTTAGACGATTCAAAAGGTGAATT
GACATCCTTTTTAGGAGCTGAAATTGCAGAATATGTAAAAAAAAATAAGCCTTC
AATAGAAAGCAAATGTTAA.

In some embodiments, a Gsh1 protein comprises a sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the sequence of SEQ ID NO: 1, or a conservatively substituted version thereof. In some embodiments, a Gsh1 protein comprises a conservatively substituted version of SEQ ID NO: 1. In some embodiments, a GSH1 gene encoding a Gsh1 protein comprises a sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the sequence of SEQ ID NO: 2.

As used herein, sequence identity refers to a measurement of the similarity between two or more sequences (e.g., nucleotide sequences and/or amino acid sequences). For example, when two or more nucleotide sequences are aligned, sequence identity refers to the number of positions within the alignment where the nucleotide is identical between the nucleotide sequences being aligned (optionally taking into account potential gaps in either sequence). When two or more amino acid sequences are aligned, sequence identity refers to the number of positions within the alignment where the amino acid is identical between the amino acid sequences being aligned (optionally taking into account potential gaps in either sequence). As one of ordinary skill in the art would appreciate, sequence identity can be determined using any of the algorithms known in the art and using default parameters (e.g., the local homology algorithm of Smith and Waterman (1970) Adv. Appl. Math. 2:482c; the algorithm of Needleman and Wunsch, J. Mol. Biol. (1970) 48:443; the method of Pearson and Lipman. Proc. Natl. Acad. Sci. USA (1998) 85:2444; BLAST, Clustal Omega, or any other sequence alignment algorithms known in the art). In some embodiments an algorithm is used that provides a local alignment. In some embodiments, an algorithm is used that provides a global alignment (e.g., the algorithm of Needleman and Wunsch). In some embodiments, sequence being aligned are the same length. In other embodiments, sequences being aligned are of different lengths. In some embodiments, sequence identity is calculated relative to the longer of the sequences being aligned, while in other embodiments, sequence identity is calculated relative to the shorter of the sequences being aligned. In some embodiments, sequence identity is calculated over an entire sequence, while in other embodiments, sequence identity is calculated over a portion of a sequence.

### Gsh1 Variants

A point mutation at cysteine 266 in the Gsh1 protein that can reduce the level of feedback inhibition by GSH has previously been reported (Biterova et al. J Biol Chem. 2010 May 7; 285(19): 14459-14466).

As described in Example 1, genome editing technology and pooled growth assays were used to systematically identify new variants of Gsh1 that confer resistance to inhibition by GSH while maintaining high enzymatic activity. The term “variant,” as used herein, refers to a nucleotide or amino acid sequence that includes at least one modification compared to a reference sequence, such as a wild-type nucleotide or amino acid sequence. Modifications can include deletions, additions, and/or substitutions of one or more nucleotides and/or amino acids. It should be understood that when a modification is described at a specific position relative to a reference sequence (e.g., SEQ ID NO: 1), one of ordinary skill in the art would be able to identify the corresponding position in a different sequence (e.g., a related or similar sequence) by aligning the different sequence with the reference sequence (e.g., SEQ ID NO: 1).

In some embodiments, a variant comprises a single amino acid change relative to a reference sequence. In some embodiments, the single amino acid change is an amino acid substitution or deletion. In other embodiments, a variant comprises more than one amino change relative to a reference sequence. In some embodiments, a variant comprises more than one amino acid substitution and/or more than one amino acid deletion relative to a reference sequence. An amino acid substitution can be a conservative substitution or a non-conservative substitution. A “conservative” amino acid substitution, as used herein, refers to an amino acid substitution that does not alter the relative charge or size characteristics of the protein in which the amino acid substitution is made. Conservative substitutions of amino acids include substitutions made amongst amino acids within the following groups: (a) methionine (M), isoleucine (I), leucine (L), and valine (V); (b) phenylalanine (F), tyrosine (Y), and tryptophan (W); (c) lysine (K), arginine (R), and histidine (H); (d) alanine (A) and glycine (G); (e) serine (S) and threonine (T); (f) glutamine (Q) and asparagine (N); and (g) glutamate (E) and aspartate (D). In some embodiments, an amino acid substitution comprises a non-conservative amino acid substitution. In some embodiments, a non-conservative amino acid substitution alters the relative charge or size characteristics of a Gsh1 protein.

In some embodiments, a Gsh1 variant comprises one or more amino acid substitutions relative to the sequence of SEQ ID NO: 1. In some embodiments, a Gsh1 variant that comprises one or more amino acid substitutions relative to the sequence of SEQ ID NO: 1 confers reduced affinity of Gsh1 for GSH. The term “affinity,” as used herein, refers to a binding ability of a molecule. e.g., a Gsh1 protein to a binding partner, e.g., a GSH molecule, where the degree of binding enables the molecule to be used to distinguish the binding partner from an appropriate control in a binding assay or other binding context. The term “reduced affinity.” as used herein refers to a binding of a molecule. e.g., a Gsh1 variant to a binding partner, e.g., a GSH molecule that is lower than the binding of a control protein, e.g., wild-type Gsh1 comprising the sequence of SEQ ID NO: 1 to a GSH molecule. In some embodiments, affinity of a Gsh1 variant to GSH is determined using buthionine sulfoximine (BSO). BSO is a competitive inhibitor of Gsh1 that binds to many of the same amino acid residues in the Gsh1 protein that are bound by GSH. Without wanting to be bound by theory, it is hypothesized that amino acid changes that confer resistance to BSO also confer resistance to GSH. In some embodiments, a Gsh1 variant that comprises one or more amino acid substitutions relative to the sequence of SEQ ID NO: 1 confers reduced binding to BSO, referred to herein as “BSO resistance,” which is indicative of reduced binding to GSH and reduced feedback inhibition of a Gsh1 variant by GSH.

In some embodiments, a Gsh1 variant that comprises one or more amino acid substitutions relative to the sequence of SEQ ID NO: 1 has reduced binding to GSH.

In some embodiments, a Gsh1 variant that comprises one or more amino acid substitutions relative to the sequence of SEQ ID NO: 1 maintains enzymatic activity of Gsh1. The phrase “maintains enzymatic activity,” as used herein, refers to an enzymatic activity of a Gsh1 variant described herein that is not substantially different from the enzymatic activity of a wild-type Gsh1 comprising the sequence of SEQ ID NO: 1. In some embodiments, an enzymatic activity is inferred using a cell-based assay. In some embodiments, an enzymatic activity is determined in the presence of an inhibitor, e.g., a toxin. In some embodiments, an enzymatic activity is determined in the presence of, e.g., cadmium chloride or ticlatone. For example, in the presence of a toxin, e.g., cadmium chloride or ticlatone a yeast cell is dependent on the presence of GSH for survival. In some embodiments, a variant Gsh1 protein maintains enzymatic activity in the presence of a toxin, e.g., continues to produce GSH and ensures survival of the yeast cell. In contrast, a yeast cell comprising a wild-type Gsh1 protein of SEQ ID NO: 1 may not produce sufficient GSH in the presence of a toxin and the yeast cells comprising a wild-type Gsh1 protein may not survive.

In some embodiments, a Gsh1 variant comprises at least one amino acid substitution relative to the sequence of SEQ ID NO: 1 at one or more of the amino acid positions listed in Table 1.

In some embodiments, a Gsh1 variant that comprises one or more amino acid substitutions relative to the sequence of SEQ ID NO: 1 confers reduced affinity of Gsh1 for GSH and maintains enzymatic activity of Gsh1, e.g., ensures survival of a yeast cell comprising the Gsh1 variant in the presence of a toxin.

In some embodiments, a Gsh1 variant that comprises two amino acid substitutions relative to the sequence of SEQ ID NO: 1 confers reduced affinity of Gsh1 for GSH and maintains enzymatic activity of Gsh1.

In some embodiments, a Gsh1 variant that comprises three amino acid substitutions relative to the sequence of SEQ ID NO: 1 confers reduced affinity of Gsh1 for GSH and maintains enzymatic activity of Gsh1.

In some embodiments, a Gsh1 variant that comprises four amino acid substitutions relative to the sequence of SEQ ID NO: 1 confers reduced affinity of Gsh1 for GSH and maintains enzymatic activity of Gsh1.

In some embodiments, a Gsh1 variant comprises at least one amino acid substitution relative to the sequence of SEQ ID NO: 1 at one or more of the amino acid positions listed in Table 1. In some embodiments, a Gsh1 variant comprises more than one amino acid substitution relative to the sequence of SEQ ID NO: 1 at the amino acid positions listed in Table 1. In some embodiments, a Gsh1 variant comprises 2, 3, 4, 5, 6, 7, 8, 9, 10, 11, 12, 13, 14, 15, 16, 17, 18, 19, 20 or more than 20 amino acid substitutions relative to the sequence of SEQ ID NO: 1 at amino acid positions listed in Table 1.

In some embodiments, a Gsh1 variant comprises an amino acid sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises at least one amino acid substitution relative to the sequence of SEQ ID NO: 1 at one or more of the amino acid positions listed in Table 1.

In some embodiments, a Gsh1 variant comprises an amino acid sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 and comprises more than one amino acid substitution relative to the sequence of SEQ ID NO: 1 at the amino acid positions listed in Table 1.

In some embodiments, a Gsh1 variant comprising at least one amino acid substitution relative to the sequence of SEQ ID NO: 1 at the amino acid positions listed in Table 1 confers reduced affinity of Gsh1 for GSH and maintains enzymatic activity of Gsh1 in the presence of a chemical stressor, e.g., toxin. In some embodiments, a Gsh1 variant comprising more than one amino acid substitution relative to the sequence of SEQ ID NO: 1 at the amino acid positions listed in Table 1 confers reduced affinity of Gsh1 for GSH and maintains enzymatic activity of Gsh1 in the presence of a chemical stressor, e.g., toxin.

In some embodiments, a Gsh1 variant comprises an amino acid substitution at one or more of the following amino acid positions relative to SEQ ID NO: 1: 24, 26, 28, 30, 32, 33. 34, 35, 36, 37, 38, 39, 40, 41, 42, 44, 45, 46, 53, 55, 59, 60, 71, 78, 79, 87, 90, 102, 107, 108, 117, 118, 121, 123, 135, 136, 137, 138, 139, 142, 144, 145, 146, 147, 151, 161, 165, 172, 174, 176, 177, 185, 198, 199, 202.203, 206.210, 220.225, 229.235, 236.238, 239.240, 242, 243, 245, 246, 247, 248, 249, 250, 251, 252, 253, 264, 265, 266, 269, 271, 276, 277, 278, 281.282, 283.285, 286.287, 288.289, 290.294, 295.296, 297.300, 301.302, 303, 304, 306, 307, 309, 310, 312, 314, 315, 316, 317, 318, 319, 321, 323, 330, 332, 335, 339, 340, 341, 348, 349, 352, 354, 358, 363, 364, 365, 366, 375, 376, 378, 379, 381, 383, 385, 386, 388, 391, 395, 398, 400, 401, 404, 406, 410, 413, 414, 422, 429, 430, 431, 432, 434, 436,443, 450, 452, 454, 455, 456, 457, 458, 460, 461, 462, 464, 465, 469, 491, 495, 497, 498, 499, 500, 501, 504, 514, 515, 517, 518, 519, 527, 528, 529, 535, 536, 538, 540, 542, 544, 545, 547, 548, 549, 550, 551, 552, 556, 557, 563, 564, 567, 568, 571, 576, 579, 581, 584.585, 586.587, 588.589, 590.594, 597.600, 603.604, 608.609, 612.616, 617.618, 619, 631, 633, 635, 637, 640, 641, 642, 643, 655, 656, 657, 664, 665, 668, 669 and/or 670.

In some embodiments, a Gsh1 variant comprises one or more of the following amino acid substitutions relative to the sequence of SEQ ID NO: 1 D24Q, G26S, E28V. L30F, L30V, Y32M, 133A, F34M, Q35L. A361, A37W, G38D, K39E, K39L, K39T, R40N, D41E. N42V, N42Y, P44V, L45F, F46C, F46E, Y53M, V55Y, D59E, D59L, D60K, H71C, L78G, N79G, E87M, D90S, L102F, A107D. S108Q, SI08K. S108C, Y117T, Y117L, Y117Q, VI 181. N121S, Q123M, E135P. Y136W, Y136C. Y136V, Y136F. A137T. A137F, A137V, R138V, R138T, R138N, Q139K, K142E, K142Q, NI44T, L145P, H146A, V147T, S151P. R161L. R161V, P165H, P165T, D172Y. W174H, H176R, H176Y, K177H, F185Y, P198E, N199T, A202H, S203L, T206F, T206R, E210S, K220E, K220T, K220S, K220L, K220N, P225S, D229W, D235K. D235V, W236E, L238H, L238I. P239K, E240F, K242A, K242W, E243L. K245S, L246P, L246K, A247S, S248F. S248L, K249M, K249C, P250F. P250Y, P250Q, P250N, G251W, F252K. I253V, C264L, C264G, C264T, C264M, C264N, S265L, S265C, S265T, C266A, C266S, V269H, V269C, V269A, F271V, I276T, I276F, I276A, N277A, N277D, N277K, N277Q, N277I, K278Q. K278A, Y281E, Y281A, Y281K, L282I, L282C, Y283H, A285N, L286M, V287A, N288T, N288L, N288I, F289L, F289Y. A290T, L294A, A295V, F296Q, F296G, F296T. S297T, A300C. P301V, A302N. A302V, A302F, A302C, F303Y, K304R, W306T, W306L, W306Y, W306M, L307I. L307V, D309G, D309Q, D309N, Q310D, Q310T, Q310N, Q310L. Q310S, V312I, W314F, N315I, N315K, V316C, V316T, V316M, 1317L, 1317T, 1317V, S318M, S318L. S318N, S318G. S318C, S318R, G319R, G319M, G319T, G319K, V321T, D323C, G330A, A332Y, A332C, L335D, N339M, K340G, N341C. K348V, D349C, D3491, D352W, D352V, V354T, P358H, S363A, S364M, V365A, D366I, F375Y, N376H, N376Q, T378G. Y379L, D381N, N383F, P385C, I386Y, 1386T, 1386S, E388F, E388A, L391C. L395A, L395Y, D398P, A400R, P401L, Y404L, Y404H, L406M, F410L. F410V, L413M, Y414C, Y414A, F422I, D429K, N430H, K431V, K431N, T432A, T432W, S434C, H436Q, T443S, F450I, P452I, T454W, Q455K, Q455H, Q456D, A457N, T458K, D460G, K461A. K461N, K462P, S464V, P465T, P465S. V469L, Y491L. D495E, I497Q, I497Y. L498I, T499V, F500K, F500E, S501R, I504N, I504Y, W514F, W514T, W514G, W5141, E515H, E515F, E515G, M517N, K518L, 1519H, L527E. F528N, F528I, E529L, K535C, S536H, 5536G, R538E, D540H, D542R, E544Q, E544D, T5451. T545S, D547V, D547M. Y548R, Y548Q, Y548S, S549F, 1550G, S551, E552R. N556H, N556M, P557M, P563M, Q564C, T567G, P568E. C571T, V576C, D579C, K581L, K581Q, K584F, H585M, H585T, S586Q, S587G, S587D, S587F, K588E, K588T, H589A, E590P, Y594S, Y594C. K597S, S600Y. S600H, A603C, S604H, S604N, P608F, T609V, K612C, N616I, N616V, F617C, V618L, L619W, S631I, S631F, S633M, S633R. S633W, N635S, D637F, S6401, T641A, C642G, D643F, D643Y, D643M, L6551, T656I. S657N, A664F, E665H, K668I, K668W, K669D and/or N670I.

In some embodiments, a Gsh1 variant comprises the following amino acid substitutions relative to the sequence of SEQ ID NO: 1: C264L:C266A:Q310D:V312I; K220E:C264L:C266A:Q310D; L282I:Q310D:V312I; K220E:C264L:L282I:L307I; K220E:C266A:Q310D:G319R; L307I:V312I:G319R; L238H:C264L:Q310D:G319R; C266A:Q310D:V312I:G319R; C264L:C266A:L282I:Q310D; C264L:N277D:L282I:Q310D; K220E:C264L:C266A:L307I; C266A:N277D:Q310D:V312I; L238H:L282I:Q310D:V312I; C264L:C266A:L282I:L307I; K220E:L238H:C264L:G319R; C266A:N277D; K220E:L238H:V312I:G319R; K220E:C264L:C266A:V312I; L282I:Q310D:G319R; C266A:L307I:Q310D; K220E:C264L:Q310D: L238H:C264L:C266A:N277D; C266A:L282I:Q310D:G319R; K220E:C266A:N277D:L282I; K220E:G319R; K220E:L238H:Q310D:G319R; K220E:L238H:C266A:N277D; C266A:N277D:L282I; L238H:C266A:L282I:Q310D; L238H:C264L:C266A:L307I; L238H:C266A:Q310D:V312I; C264L:C266A:L282I:V312I; K220E:C264L:C266A; K220E:C264L:L307I; K220E:C264L:N277D:Q310D; L238H:C266A:Q310D:G319R; L238H:C264L:C266A:Q310D; K220E:N277D:L282I:Q310D; C264L:C266A:N277D:L307I; K220E:C266A:L282I:Q310D; N277D:L282I:Q310D:G319R; C264L:C266A:Q310D; L238H:C266A:N277D:L282I; K220E:C266A:L307I:V312I; L238H:C264L:C266A:L282I; C264L:C266A:N277D:Q310D; K220E:N277D:Q310D:V312I; C264L:N277D:Q310D:G319R; K220E:L238H:C266A:Q310D; L238H:Q310D:G319R; C264L:C266A:L307I:V312I; K220E:N277D:Q310D:G319R; K220E:L238H:L282I:V312I; C264L:L282I:G319R; K220E:L282I:G319R; C266A:L282I:G319R; K220E:C264L:L307I:V312I; K220E:C266A:V312I:G319R; K220E:L307I:V312I; K220E:N277D:L282I:G319R; K220E:C266A:N277D; C266A:N277D:L307I; C266A:N277D:L282I:V312I; C266A:Q310D:V312I; K220E:C266A:Q310D:V312I; K220E:C266A:L307I:G319R; C264L:C266A:N277D; C264L:C266A:L282I:G319R; L238H:C264L:G319R; C264L:N277D:V312I:G319R: L238H:C266A:L282I:V312I; K220E:C266A:L282I:L307I; C266A:L307I:V312I; C266A:L282I:V312I; K220E:C264L:C266A:G319R; C264L:C266A:V312I:G319R; L238H:C264L:C266A; C264L:N277D:Q310D; L238H:C264L:L282I; K220E:C266A; L238H:C266A:N277D; K220E:C266A:V312I; L238H:C266A:L307I; K220E:C266A:N277D:L307I; L238H:C266A:V312I; K220E:C266A:L282I: L238H:C266A:Q310D; K220E:L238H:C264L:V312I; C266A:G319R; K220E:C266A:L307I: L238H:N277D:V312I:G319R; L238H:Q310D; C266A:L282I:L307I; L238H:C266A:L282I:G319R; C266A:Q310D:G319R; L238H:N277D:Q310D: L238H:C264L:N277D:G319R; C266A:L282I; K220E:C266A:L282I:G319R; C266A:N277D:Q310D:G319R; K220E:L238H:Q310D; C266A:N277D:G319R; L238H:L282I:L307I:G319R; L238H:C266A; K220E:C266A:G319R; L238H:N277D:L282I:G319R; L238H:C264L:C266A:G319R; C266A:N277D:Q310D; L238H:C266A:N277D:V312I; C266A:L307I; C264L:C266A:N277D:V312I; C266A:N277D:L307I:V312I; C264L:C266A; C264L:C266A:G319R; L238H:C266A:N277D:L307I; K220E:L238H:L307I:G319R; L238H:C266A:G319R; K220E:N277D:V3I21:G319R; V312I:G319R; K220E:C266A:N277D:G319R; K220E:C264L; L238H:C266A:L282I:L307I; L238H:L282I:Q310D; C264L:N277D:V312I; K220E:L238H:C266A:V312I; C264L:C266A:L307I; L238H:C266A:N277D:G319R; K220E:L238H:C266A; C266A:Q310D; L238H:C266A:L282I; L238H:C266A:L307I:G319R; C266A:L307I:G319R; K220E:N277D:G319R; C266A:N277D:V312I; L238H:V312I: L238H:C264L:C266A:V312I; C264L:C266A:L307I:G319R; K220E:C264L:V312I:G319R; N277D:L282I:L307I:G319R; or L238H:N277D:L282I:V312I.

It should be appreciated that other gamma glutamylcysteine synthetase enzymes are also compatible with aspects of the disclosure. One of ordinary skill in the art would be able to identify amino acids in another gamma glutamylcysteine synthetase enzymes that correspond to specific positions in SEQ ID NO: 1 by aligning the sequence of the other gamma glutamylcysteine synthetase enzymes with the sequence of SEQ ID NO: 1, and one of ordinary skill in the art would accordingly be able to introduce amino acid substitutions in other gamma glutamylcysteine synthetase enzymes at one or more positions corresponding to any of the positions disclosed herein for SEQ ID NO: 1.

In some embodiments, a Gsh1 variant described herein when expressed in a yeast cell confers resistance of the yeast cell to chemical stressors including toxins, e.g., cadmium chloride or ticlatone, and maintains enzymatic activity. For example, in some embodiments, a Gsh1 variant described herein may have an enzymatic activity that is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97% or at least 99% of the enzymatic activity of a Gsh1 protein comprising the sequence of SEQ ID NO: 1.

In some embodiments, a Gsh1 variant described herein has reduced affinity for GSH. In some embodiments, a Gsh1 variant binds to GSH with reduced affinity relative to a wildtype Gsh1 protein, such as a Gsh1 protein comprising the sequence of SEQ ID NO: 1. In some embodiments, a Gsh1 variant described herein binds to GSH with a dissociation constant (KD) of more than about 10⁻¹³ M, 10⁻¹² M, 10⁻¹¹ M, 10⁻¹⁰ M, 10⁻⁹ M, 10⁻⁸ M, 10⁻⁷ M, 10⁻⁶ M, 10⁻⁵ M, 10⁻⁴ M, 10⁻³ M, 10⁻² M, 10⁻¹ M or more. In some embodiments, a Gsh1 variant described herein binds to GSH with a dissociation constant (KD) of less than about 10⁻¹³ M, 10⁻¹² M, 10⁻¹¹ M, 10⁻¹⁰ M, 10⁻⁹ M, 10⁻⁸ M, 10⁻⁷ M, 10⁻⁶ M, 10⁻⁵ M, 10⁻⁴ M, 10⁻³ M, 10⁻² M, 10⁻¹ M or less. The term “about.” as used herein refers to a value that is similar to a stated reference value and includes values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

In some embodiments, a Gsh1 variant described herein confers resistance to BSO. In some embodiments, a Gsh1 variant described herein confers resistance to BSO and maintains enzymatic activity. In some embodiments, a Gsh1 variant described herein binds to BSO with a dissociation constant (KD) of more than about 10⁻¹³ M, 10⁻¹² M, 10⁻¹¹ M, 10⁻¹⁰ M, 10⁻⁹ M, 10⁻⁸ M, 10⁻⁷ M, 10⁻⁶ M, 10⁻⁵ M, 10⁻⁴ M, 10⁻³ M, 10⁻² M, 10⁻¹ M or more. In some embodiments, a Gsh1 variant described herein binds to GSH with a dissociation constant (KD) of less than about 10⁻¹³ M, 10⁻¹² M, 10⁻¹¹ M, 10⁻¹⁰ M, 10⁻⁹ M, 10⁻⁸ M, 10⁻⁷ M, 10⁻⁶ M, 10⁻⁵ M, 10⁻⁴ M, 10⁻³ M, 10⁻² M, 10⁻¹ M or less.

In some embodiments, a GSH1 feedback resistant allele is in the S. cerevisiae GSH1 gene. In other embodiments, a GSH1 feedback resistant allele is in a gene derived from a different organism and the allele is heterologously expressed in a yeast host cell, such as a S. cerevisiae host cell. Methods of introducing heterologous sequences into a yeast host cell are well-known in the art.

### Bifunctional GCS-GS

Aspects of the disclosure relate to heterologous expression of a bifunctional gamma-glutamylcysteine synthetase-glutathione synthetase (GCS-GS). As used herein, a “bifunctional heterologous gamma-glutamylcysteine synthetase-glutathione synthetase” or “GCS-GS” refers to an enzyme that encodes both glutamylcysteine synthetase and glutathione synthetase activities for GSH biosynthesis (see e.g., Gopal et al., 2005, Janowiak and Griffith, 2005, Vergauwen et al., 2006). In bacteria, a GCS-GS produces GSH. In some embodiments, a GCS-GS is resistant to feed-back inhibition by GSH.

In some embodiments, a yeast host cell described herein expresses a bifunctional heterologous GCS-GS enzyme derived from bacteria. In some embodiments, a yeast host cell described herein expresses a bifunctional heterologous GCS-GS enzyme derived from S. thermophilus or S. agalactiae. In some embodiments, a yeast host cell described herein comprises a bifunctional heterologous GCS-GS enzyme derived from another bacterial species.

Advantageously, a bifunctional heterologous GCS-GS enzyme derived from S. thermophilus or S. agalactiae is innately resistant to feedback inhibition. In some embodiments, a yeast host cell described herein comprises a bifunctional heterologous GCS-GS enzyme derived from S. thermophilus. In some embodiments, a yeast host cell described herein comprises a bifunctional heterologous GCS-GS enzyme derived from S. agalactiae. Heterologous sequences encoding bifunctional heterologous GCS-GS enzymes, such as heterologous GCS-GS enzymes derived from S. thermophilus or S. agalactiae can be introduced into yeast cells using methods known in the art.

In some embodiments, a yeast host cell described herein comprises a bifunctional heterologous GCS-GS enzyme derived from S. thermophilus and a Gsh1 variant as described herein. In some embodiments, a yeast host cell described herein comprises a bifunctional heterologous GCS-GS enzyme derived from S. agalactiae and a Gsh1 variant as described herein. In some embodiments, a yeast host cell described herein comprises a bifunctional heterologous GCS-GS enzyme derived from S. thermophilus, a bifunctional heterologous GCS-GS enzyme derived from S. agalactiae, and a Gsh1 variant as described herein.

In some embodiments, a GCS-GC protein comprises the sequence of SEQ ID NO: 3:

(SEQ ID NO: 3)
MTLNQLLQKLEATSPILQANFGIERESLRVDRQGQLVHTPHPSCLGARSFHPYIQTDF
CEFQMELITPVAKSTTEARRFLGAITDVAGRSIATDEVLWPLSMPPRLKAEEIQVAQL
ENDFERHYRNYLAEKYGTKLQAISGIHYNMELGKDLVEALFQESDQTDMIAFKNAL
YLKLAQNYLRYRWVITYLFGASPIAEQGFFDQEVPEPVRSFRNSDHGYVNKEEIQVS
FVSLEDYVSAIETYIEQGDLNAEKEFYSAVRFRGQKVNRSFLDKGITYLEFRNFDLNP
FERIGISQTTMDTVHLLILAFLWLDSPENVDQALAQGHALNEKIALSHPLKPLPSEAK
TQDIVTALDQLVQHFGLGDYHQDLVKQVKAAFADPNQTLSAQLLPYIKDKSLAEFA
LNKALAYHDYDWTAHYALKGYEEMELSTQMLLFDAIQKGIHFEILDEQDQFLKLW
HQDHVEYVKNGNMTSKDNYVVPLAMANKTVTKKILADAGFSVPSGDEFTSLEEGL
AYYPLIKDKQIVVKPKSTNFGLGISIFQEPASLDNYQKALEIAFAEDTSVLVEEFIPGTE
YRFFILDGRCEAVLLRVAANVIGDGKHTIRELVAQKNANPLRGRDHRSPLEIIELGDI
EQLMLAQQGYTPDDILPEGKKVNLRRNSNISTGGDSIDVTETMDSSYQELAAAMAT
SMGAWACGVDLIIPDETQIATKENPHCTCIELNFNPSMYMHTYCAEGPGQAITTKILD
KLFPEIVAGQT.

In non-limiting embodiments, the GCS-GC protein comprising the sequence of SEQ ID NO: 3 may be encoded by a polynucleotide comprising the sequence of SEQ ID NO: 4:

(SEQ ID NO: 4)
ATGACTTTGAACCAATTGTTGCAAAAGCTGGAAGCTACTTCTCCAATCTTACAGG
CCAACTTCGGTATTGAAAGAGAATCTTTACGTGTTGACAGACAAGGTCAATTGGT
TCACACCCCACACCCATCCTGTTTGGGTGCCAGATCATTCCACCCATACATCCAA
ACTGACTTCTGTGAATTCCAAATGGAATTAATCACTCCAGTTGCCAAATCTACCA
CAGAAGCTAGAAGATTTTTGGGTGCCATTACTGATGTTGCCGGTAGATCCATAGC
TACTGATGAAGTCTTATGGCCACTGTCCATGCCACCAAGATTGAAAGCTGAAGA
AATCCAAGTCGCTCAATTAGAAAACGATTTTGAAAGACATTACAGAAACTATTT
GGCTGAAAAGTACGGTACCAAGCTACAAGCCATCTCCGGTATTCACTACAATAT
GGAATTGGGTAAAGATTTGGTCGAAGCCTTGTTCCAAGAATCCGACCAAACCGA
CATGATTGCTTTCAAGAACGCCTTGTACTTGAAGTTGGCCCAAAACTACTTAAGA
TACAGATGGGTTATCACTTATTTGTTTGGTGCTTCTCCAATTGCCGAACAAGGTTT
CTTTGACCAAGAAGTTCCAGAACCAGTCAGATCATTCAGAAATTCTGACCACGG
TTACGTTAATAAGGAAGAAATTCAAGTTTCCTTCGTTTCTCTCGAAGATTACGTT
TCTGCCATCGAAACCTATATTGAACAAGGCGATTTGAACGCCGAAAAAGAATTC
TACTCCGCTGTCAGGTTCAGAGGTCAAAAGGTTAACAGATCTTTCTTGGACAAAG
GCATTACCTACTTGGAATTCAGAAACTTCGATCTTAACCCTTTCGAAAGAATCGG
TATCTCTCAAACCACCATGGACACTGTCCACTTGCTTATCTTAGCTTTCTTATGGT
TGGATTCTCCAGAAAACGTCGACCAAGCATTGGCTCAAGGTCACGCTTTGAACG
AAAAGATTGCTTTATCTCATCCATTGAAGCCATTGCCGTCTGAAGCTAAGACCCA
AGATATTGTCACCGCTTTGGACCAATTGGTCCAACACTTCGGTTTAGGTGATTAC
CATCAAGACTTGGTTAAGCAAGTTAAGGCCGCCTTTGCAGACCCTAACCAAACG
TTATCCGCTCAACTGTTGCCATACATCAAGGATAAGTCTTTGGCTGAATTTGCTTT
GAACAAGGCTTTGGCTTACCACGACTACGACTGGACTGCTCACTACGCTTTGAAG
GGTTACGAAGAAATGGAATTGTCCACTCAAATGTTGTTGTTCGACGCTATCCAAA
AGGGTATTCATTTCGAGATCTTGGATGAACAAGATCAATTCCTCAAGTIGTGGCA
CCAAGATCACGTTGAATACGTGAAGAACGGTAACATGACCTCCAAGGACAATTA
TGTTGTTCCATTAGCTATGGCTAACAAGACTGTTACCAAGAAGATCTTGGCTGAC
GCTGGTTTCTCTGTCCCATCTGGTGATGAATTCACTTCTTTGGAAGAAGGTTTGG
CTTACTACCCATTGATTAAAGACAAGCAAATCGTTGTCAAGCCAAAGAGCACCA
ACTTCGGTTTGGGTATTTCCATTTTTCAAGAACCAGCTTCTTTGGACAACTACCA
AAAGGCTTTGGAAATCGCTTTCGCTGAAGACACCTCAGTTTTGGTCGAAGAATTC
ATTCCAGGTACTGAATACAGATTCTTCATCCTAGACGGTAGATGTGAAGCCGTTT
TGTTGAGAGTTGCTGCTAACGTTATCGGTGATGGTAAGCACACTATCCGTGAATT
GGTCGCTCAAAAAAATGCTAACCCATTAAGAGGTCGTGATCACCGTTCCCCATTG
GAAATTATTGAATTGGGAGACATAGAACAATTGATGTTGGCTCAACAAGGTTAC
ACCCCAGATGACATCTTGCCAGAAGGTAAGAAGGTCAACTTGAGAAGAAACTCT
AACATCTCTACTGGTGGCGACAGTATTGATGTCACCGAAACCATGGATTCTTCTT
ACCAAGAGCTAGCTGCTGCCATGGCTACCTCCATGGGTGCTTGGGCTTGTGGTGT
TGACTTGATCATCCCAGACGAAACTCAAATTGCTACCAAGGAAAACCCTCATTG
CACCTGTATCGAATTGAACTTCAACCCTTCCATGTACATGCACACTTACTGTGCT
GAAGGTCCAGGTCAAGCTATTACTACTAAGATTTTAGACAAGCTATTCCCAGAA
ATCGTTGCTGGTCAAACT.

In some embodiments, a GCS-GC protein comprises the sequence of SEQ ID NO: 5:

(SEQ ID NO: 5)
MIIDRLLQRSHSHLPILQATFGLERESLRIHQPTQRVAQTPHPKTLGSRNYHPYIQTDY
SEPQLELITPIAKDSQEAIRFLKAISDVAGRSINHDEYLWPLSMPPKVREEDIQIAQLED
AFEYDYRKYLEKTYGKLIQSISGIHYNLGLGQELLTSLFELSQADNAIDFQNQLYMK
LSQNFLRYRWLLTYLYGASPVAEEDFLDQKLNNPVRSLRNSHLGYVNHKDIRISYTS
LKDYVNDLENAVKSGQLIAEKEFYSPVRLRGSKACRNYLEKGITYLEFRTFDLNPFSP
IGITQETVDTVHLFLLALLWIDSSSHIDQDIKEANRLNDLIALSHPLEKLPNQAPVSDL
VDAMQSVIQHFNLSPYYQDLLESVKRQIQSPELTVAGQLLEMIEGLSLETFGQRQGQI
YHDYAWEAPYALKGYETMELSTQLLLFDVIQKGVNFEVLDEQDQFLKLWHNSHIE
YVKNGNMTSKDNYIVPLAMANKVVTKKILDEKHFPTPFGDEFTDRKEALNYFSQIQ
DKPIVVKPKSTNFGLGISIFKTSANLASYEKAIDIAFTEDSAILVEEYIEGTEYRFFVLE
GDCIAVLLRVAANVVGDGIHTISQLVKLKNQNPLRGYDHRSPLEVIELGEVEQLMLE
QQGYTVNSIPPEGTKIELRRNSNISTGGDSIDVTNTMDPTYKQLAAEMAEAMGAWV
CGVDLIIPNATQAYSKDKKNATCIELNFNPLMYMHTYCQEGPGQSITPRILAKLFPEL.

In non-limiting embodiments, the GCS-GC protein comprising the sequence of SEQ

ID NO: 5 may be encoded by a polynucleotide comprising the sequence
of SEQ ID NO: 6
(SEQ ID NO: 6)
ATGATCATTGACAGATTATTACAAAGATCTCACTCTCATTTGCCAATCTTGCAAG
CTACTTTCGGTTTGGAAAGAGAATCCTTGCGTATCCACCAACCAACCCAAAGAGT
TGCCCAAACCCCTCACCCAAAGACCTTGGGTTCTAGAAACTACCACCCATACATT
CAAACTGACTACTCTGAACCACAACTCGAGCTAATCACACCAATTGCCAAGGAT
TCTCAAGAAGCGATCCGTTTCTTGAAGGCCATTTCTGATGTCGCCGGTAGATCCA
TTAACCATGATGAATATCTATGGCCATTGTCCATGCCACCTAAGGTCCGTGAAGA
AGACATACAAATTGCTCAACTGGAAGATGCTTTCGAATACGATTACAGAAAGTA
CTTAGAAAAAACTTACGGCAAATTGATCCAATCCATCTCTGGTATCCACTACAAC
TTGGGCTTGGGTCAAGAATTATTGACTTCTTTGTTTGAATTGTCTCAAGCTGATA
ACGCTATTGATTTCCAAAACCAACTTTACATGAAGTTGAGTCAAAATTTTTTGAG
ATACAGATGGTTGTTGACCTATTTGTACGGTGCTTCCCCAGTTGCTGAAGAAGAC
TTCTTAGACCAAAAGCTCAACAACCCAGTCAGATCGCTTCGTAATTCACACCTAG
GTTACGTCAACCACAAGGACATTAGAATTTCTTACACTTCCTTGAAAGACTACGT
TAATGACTTAGAAAACGCTGTCAAGAGTGGTCAATTGATCGCCGAAAAAGAATT
CTACTCCCCAGTCAGATTGCGTGGTTCCAAGGCTTGTAGAAACTATTTGGAAAAG
GGTATTACTTACTTGGAGTTCAGAACTTTTGATTTGAACCCATTCTCCCCAATTGG
TATCACTCAAGAAACCGTCGACACCGTTCACTTGTTCTTGTTAGCCCTATTATGG
ATTGACTCTTCTTCTCACATTGATCAAGACATCAAGGAAGCCAACCGGTTGAACG
ATCTAATCGCTTTGTCCCATCCACTCGAAAAATTGCCAAACCAAGCCCCAGTCTC
TGACTTAGTCGATGCCATGCAATCTGTTATCCAACATTTCAACTTGTCCCCTTACT
ACCAAGACTTGTTGGAATCTGTGAAGAGACAAATTCAATCTCCAGAATTGACTG
TAGCTGGTCAATTGTTAGAAATGATTGAAGGTTTGTCTTTGGAAACTTTCGGTCA
AAGACAAGGTCAAATTTACCACGACTACGCTTGGGAAGCTCCATACGCTTTGAA
GGGTTACGAAACCATGGAATTGTCCACTCAATTGTTGTTGTTCGACGTTATCCAA
AAGGGTGTCAACTTCGAAGTCTTGGACGAACAAGACCAATTCTTAAAGTTGTGG
CACAACTCTCACATCGAATACGTTAAGAACGGTAACATGACTTCTAAGGACAAC
TATATTGTTCCATTGGCTATGGCTAACAAGGTTGTGACTAAGAAGATTTTGGATG
AAAAGCACTTCCCAACCCCATTCGGTGACGAATTCACCGACAGAAAGGAAGCTT
TGAACTACTTCTCTCAAATCCAAGATAAGCCAATTGTCGTTAAGCCAAAGTCTAC
TAACTTCGGTTTGGGTATTTCTATTTTCAAGACTTCTGCTAACTTGGCCTCCTACG
AAAAGGCAATTGATATCGCTTTCACTGAAGATTCCGCTATCCTGGTCGAAGAATA
CATCGAAGGTACCGAATACAGATTCTTTGTCTTGGAAGGTGACTGTATTGCTGTT
TTGTTGAGAGTTGCTGCTAACGTTGTTGGTGATGGTATCCACACTATTTCACAGT
TGGTTAAACTGAAGAACCAAAACCCGTTGAGAGGTTACGACCACAGATCTCCAT
TGGAAGTTATTGAGTTGGGTGAAGTTGAACAATTGATGTTGGAACAACAAGGCT
ATACTGTTAATTCAATCCCACCAGAAGGTACTAAGATCGAACTAAGAAGAAACT
CCAACATCTCTACTGGTGGTGATAGTATTGATGTTACCAACACCATGGACCCAAC
TTACAAGCAACTAGCTGCTGAAATGGCTGAAGCTATGGGTGCTTGGGTTTGCGGT
GTCGACTTGATCATCCCAAACGCCACCCAAGCTTACTCTAAGGACAAGAAGAAT
GCTACCTGTATCGAATTGAACTTTAACCCATTGATGTACATGCACACCTACTGTC
AAGAAGGTCCAGGTCAATCCATCACCCCAAGAATCTTGGCTAAACTTTTCCCAG
AATTA.

In some embodiments, a GCS-GC protein comprises a sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the sequence of SEQ ID NO: 3 or SEQ ID NO: 5, or a conservatively substituted version thereof. In some embodiments, a GCS-GC protein comprises a conservatively substituted version of SEQ ID NO: 3 or SEQ ID NO: 5. In some embodiments, a gene encoding a GCS-GC protein comprises a sequence that is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the sequence of SEQ ID NO: 4 or SEQ ID NO: 6.

### Cells

Further provided are microbial cells, such as yeast cells, expressing Gsh1 variants described herein and/or expressing a bifunctional heterologous GCS-GS enzyme.

Aspects of the disclosure relate to yeast cells comprising Gsh1 variants wherein the yeast cell does not comprise a new genetic element and/or new genetic material. Aspects of the disclosure relate to yeast cells comprising Gsh1 variants in which one or more amino acid substitutions is generated by gene editing of one or more nucleotide triplets encoding the amino acid sequence of Gsh1.

In some embodiments, genome editing of an endogenous Gsh1 gene is achieved via homologous recombination using a donor nucleic acid containing the nucleotide triplet that effectuates the desired amino acid change. In some embodiments, the resulting yeast cell comprises a Gsh1 variant comprising an amino acid substitution at a position disclosed in Table 1 or 2.

In some embodiments, a yeast cell is selected from the group consisting of be Saccharomyces cerevisiae, Saccharomyces cerevisiae var. chevalieri, Saccharomyces pastoriasus, Pichia spp, Saccharomyces diastaticus, Hanseniaspora uvarum, Metschnikowia pulcherrima, Pichia kudriavzevii, Torulaspora delbruekii, and Zygotorulaspora florentina, Issatchenkia orientalis, Pichia membranaefaciens, P. kudriavzevii, or Wickerhamomyces anomalus.

In some embodiments, a yeast cell is from an oleaginous yeast, such as Yarrowia lipolytica.

In some embodiments, a yeast cell is a cell of Saccharomyces cerevisiae (also known as brewer's yeast or baker's yeast). In some embodiments, a yeast cell is diploid, triploid, or tetraploid. In some embodiments, a yeast cell is a cell in an active dry yeast, an instant dry yeast, a compressed yeast, a crumbled yeast, or a cream yeast for breadmaking applications.

In some embodiments, a yeast cell is within a laboratory yeast strain. In some embodiments, a yeast cell is within an industrial yeast strain. In some embodiments, a yeast cell is within an industrial baking yeast strain. The term “yeast strain,” as used herein, refers to a yeast cell population that is genetically identical.

In some aspects, “yeast” is understood as a commercial product, such as a commercial product obtained by implementation of a method as described herein.

Yeast cells having different properties can be obtained from a single strain. For example, genome editing methods described herein can be used within many different yeast strains to allow such yeast strains to express Gsh1 variants described herein and/or bifunctional heterologous GCS-GS enzymes.

In some embodiments, a yeast strain comprising a Gsh1 variant is generated using methods, e.g., as described in Roy et al. (2018) Nature Biotechnol. 36: 512-520 or U.S. Patent Publication No. US2020/0270632, both of which are incorporated herein by reference in their entireties.

In some embodiments, a microbial cell is a bacterial cell, such as a Lactobacillus cell. It should be appreciated that other types of microbial cells, including other types of bacterial cells, may be compatible with aspects of the disclosure.

In some embodiments, a yeast cell comprising a Gsh1 variant described herein has improved GSH production compared to a control yeast cell. In some embodiments, a control yeast cell is a yeast cell that is of a same strain as a yeast cell comprising a Gsh1 variant but does not contain the Gsh1 variant. In some embodiments the control yeast cell comprises a wild-type Gsh1 protein. For example, a control yeast cell could comprise two Gsh1 alleles that encode a Gsh1 protein that comprises the sequence of SEQ ID NO: 1. In some embodiments, a yeast cell described herein comprises two Gsh1 alleles encoding a Gsh1 variant described herein. In some embodiments, a yeast cell described herein comprises one Gsh1 allele encoding a Gsh1 variant described herein and one Gsh1 allele comprising a wild-type Gsh1 protein that comprises the sequence of SEQ ID NO: 1. In some embodiments, a yeast cell comprising a Gsh1 variant described herein produces higher levels of GSH than a control yeast cell and demonstrates increased survival in the presence of a cellular stressor, e.g., cadmium chloride and ticlatone.

In some embodiments, a yeast cell expressing a Gsh1 variant described herein produces between about 1.2-fold to about 20-fold higher GSH compared to a yeast cell expressing a wild-type Gsh1 protein comprising the sequence of SEQ ID NO: 1. In some embodiments, a yeast cell expressing a Gsh1 variant described herein produces a GSH amount that is about at least 1.4-fold higher, 1.6-fold higher, 1.8-fold higher, 2-fold higher, 2.2-fold higher, 2.4-fold higher, 2.6-fold higher, 2.8-fold higher, 3-fold higher, 3.2-fold higher, 3.4-fold higher, 3.6-fold higher, 3.8-fold higher, at least 4-fold higher, at least 4.5-fold higher, at least 5-fold higher, at least 5.5-fold higher, at least 6-fold higher, at least 6.5-fold higher, at least 7-fold higher, at least 7.5-fold higher, at least 8-fold higher, at least 8.5-fold higher, at least 9-fold higher, at least 9.5-fold higher, at least 10-fold higher, at least 10.5-fold higher, at least 11-fold higher, at least 11.5-fold higher, at least 12-fold higher, at least 12.5-fold higher, at least 13-fold higher, at least 13.5-fold higher, at least 14-fold higher, at least 14.5-fold higher, at least 15-fold higher, at least 15.5-fold higher, at least 16-fold higher, at least 16.5-fold higher, at least 17-fold higher, at least 17.5-fold higher, at least 18-fold higher, at least 18.5-fold higher, at least 19-fold higher, at least 19.5-fold higher, or at least 20-fold higher, compared to a GSH amount produced by a yeast cell expressing a wild-type Gsh1 protein comprising the sequence of SEQ ID NO: 1.

It should be appreciated that any method of determining the ability of a Gsh1 variant to increase the GSH content in a yeast cell may be compatible with aspects of the disclosure. In some embodiments, the ability of a Gsh1 variant to increase the levels of GSH in a yeast cell is compared to a wild-type GSH1 gene comprising the sequence of SEQ ID NO: 2. The term “determining,” as used herein includes, e.g., measuring, detecting, and/or assaying. In some embodiments, a method of determining the ability of a Gsh1 variant to produce GSH in a feedback resistant manner is measured as the level of GSH produced by a yeast cell expressing the Gsh1 variant compared to the level of GSH produced by a yeast cell expressing Gsh1 comprising the sequence of SEQ ID NO: 1.

In some embodiments, yeast cells expressing Gsh1 variants described herein exhibit increased GSH production relative to control yeast cells. In some embodiments, yeast cells expressing Gsh1 variants described herein exhibit increased GSH production relative to control yeast cells in the presence of toxins.

Further aspects of the disclosure related to purifying GSH from yeast cells described herein using methods known in the art. In some embodiments, GSH purified from yeast cells described herein can be used for, e.g., nutraceuticals, supplements, cosmetics, skincare products, and/or pharmaceuticals. In other embodiments, yeast cells described herein can be used for yeast extracts or postbiotics with high antioxidant value for human or animal consumption. In yet other embodiments, yeast cells described herein can be used as probiotics or live yeast for producing wine or bread with high antioxidant value.

Increasing the amount of GSH produced by yeast cells would reduce the cost of making GSH by fermentation. This would directly benefit industrial applications whereby GSH must be purified from a reactor. In applications where GSH is not purified (e.g., whole cell or yeast extract products), increasing the amount of GSH would improve the antioxidant properties, and thus quality, of the product.

The GSH product produced by cells described herein can be purified at various levels of purity (e.g., 5% to 99.9% of the dry mass). The GSH product produced by cells described herein can be formulated as GSH-enriched yeast extract with concentrations of >10 g/kg of product dry mass. The GSH product produced by cells described herein can be formulated as inactivated yeast cells comprising yeast cells described herein.

### Compositions

Aspects of the disclosure relate to compositions comprising yeast cells that have increased GSH production, and compositions comprising GSH produced by such cells. Yeast cells described herein comprising Gsh1 variants may be of particular use in breadmaking applications using high-strength flour, in high-speed processes (e.g., frozen bread dough), or in applications where extensibility is especially important (e.g., pizza dough).

Aspects of the disclosure related to yeast cells and compositions comprising yeast cells, or compositions comprising GSH produced by such yeast cells, that have improved breadmaking capabilities due to increased production of GSH.

In some embodiments, provided is a composition for breadmaking comprising an inactive yeast cell comprising a Gsh1 variant described herein. In some embodiments, a composition comprises an inactive yeast cell comprising a Gsh1 variant and having a higher GSH content compared to a wild-type yeast cell.

In some embodiments, provided is a composition for breadmaking comprising purified GSH isolated from a yeast cell comprising a Gsh1 variant described herein.

In some embodiments, provided is a system for breadmaking comprising a composition comprising yeast cells that comprise a Gsh1 variant and a composition comprising yeast cells that comprise a wild-type Gsh1 protein. In some embodiments, the system for breadmaking further comprises an oxidizing agent.

Compositions described herein can include one or more of: active dry yeast, instant dry yeast, compressed yeast, crumbled yeast, cream yeast, or baker's yeast. In some embodiments, a composition is baker's dough.

In some embodiments, a breadmaking composition further comprises flour and/or salt. In some embodiments, a breadmaking composition further comprises an oil, leavening or butter. In some embodiments, a breadmaking composition further comprises sugar and/or eggs.

Further aspects of the disclosure relate to compositions comprising yeast cells comprising a Gsh1 variant described herein, which yeast cells have improved winemaking capabilities due to increased GSH production, or compositions comprising GSH produced by such cells. Yeast cells described herein comprising Gsh1 variants may be of particular use in winemaking applications to divert sulfur away from hydrogen sulfate formation and, thereby, reduce the content of volatile sulfur-containing compounds that might possess undesirable characteristics, e.g., an unpleasant smell.

Further aspects of the disclosure relate to compositions comprising yeast cells comprising a Gsh1 variant described herein, which yeast cells are useful as nutraceutical and/or pharmaceuticals due to increased GSH content, or compositions comprising GSH produced by such cells. Yeast cells described herein comprising Gsh1 variants may be of particular use in nutraceuticals and/or pharmaceuticals to increase the intake of GSH to scavenge free radicals in a subject. In some embodiments, provided are nutraceutical and/or pharmaceutical compositions comprising yeast cells comprising a Gsh1 variant described herein or comprising GSH produced by yeast cells described herein. In some embodiments, provided are nutraceutical and/or pharmaceutical compositions comprising GSH purified from yeast cells comprising a Gsh1 variant described herein.

Further aspects of the disclosure relate to compositions comprising yeast cells that have improved use in cosmetics due to increased GSH content. Yeast cells described herein comprising Gsh1 variants, or GSH produced by such cells, may be of particular use in cosmetics to protect against skin damage that leads to hyperpigmentation and wrinkles. In some embodiments, provided are cosmetic compositions comprising GSH purified from yeast cells comprising a Gsh1 variant described herein.

Further aspects of the disclosure relate to compositions comprising yeast cells that have improved use as plant protectants due to increased GSH content. Yeast cells described herein comprising Gsh1 variants, or GSH produced by such cells, may be of particular use as plant protectants to augment plant productivity and enhance crop nutrition. In some embodiments, the compositions comprising yeast cells comprising a Gsh1 variant described herein have increased GSH content and can be used during plant growth and/or after harvest. In some embodiments, provided are plant protectant compositions comprising GSH purified from yeast cells comprising a Gsh1 variant described herein.

Further aspects of the disclosure relate to compositions comprising yeast cells that have improved use in animal feed due to increased GSH content. Yeast cells described herein comprising Gsh1 variants, or GSH produced by such cells, may be of particular use in animal feed to scavenge free radicals from an animals' body and prevent or reduce damage to the animal's cells caused by free radicals. In some embodiments, provided are animal feed compositions comprising GSH purified from yeast cells comprising a Gsh1 variant described herein.

In some embodiments, a product comprising a composition described herein or fractions purified thereof at varying degrees of purity is used in applications for breadmaking, pizza, croissant, sourdough, etc., yeast extracts, food flavoring, winemaking, animal feed, cosmetics, human nutraceuticals, pharmaceuticals, and/or plant protectants.

### Methods of Use

Compositions described herein may be useful for, e.g., breadmaking, production of fermented beverages, production of nutraceuticals and/or pharmaceuticals, production of cosmetics, production of plant protectants, and/or production of animal feed.

### Breadmaking

Disulfide bonds between cysteine amino acid residues in gluten proteins play a critical role in the breadmaking process. Formation of these bonds increases dough strength, while decreasing dough extensibility. These bonds are broken down mechanically during the mixing process (e.g., when flour, water, yeast and salt are mixed to create dough); however, they can also be broken down chemically using reducing agents.

Chemical reducing agents, commonly known as “dough relaxers,” are ingredients that make dough easier to manipulate and shape. Reducing agents are especially useful in the pizza industry, for example, by preventing snapback, shrinking, or curling after the pizza has been formed. Reducing agents also have particular importance in breadmaking processes that use high-strength flour and require high-speed mixing. These agents reduce the time and energy needed for mixing. In frozen bread dough application, reducing mixing time helps preserve the stability of the yeast.

GSH is a potent natural reducing agent and yeast strains producing high concentrations of GSH can be useful, e.g., for pizza and frozen bread dough applications. For example, these strains can be used to manufacture purified GSH by fermentation. Purified GSH could then be used in a manner similar to the commonly-used reducing agent L-cysteine, that is, purified GSH could be added to flour before preparation of pizza dough. Due to its potent reducing activity, the amount of GSH could first be titrated. To this end, the GSH may first be blended with an inert ingredient (such as dairy whey) to allow precise measuring of small quantities of GSH. Inactive yeast derived from a high-GSH strain could also be used to deliver GSH reducing power to dough.

In addition, active yeast strains producing high concentrations of GSH could be used directly in breadmaking processes. For example, such strains could be used as a component of frozen dough (e.g., fresh dough that is frozen and stored before proofing and baking). Even though GSH remains inside viable yeast cells and does not react with gluten proteins in the dough, the freezing process would compromise some cells, allowing GSH to leach out and act as a reducing agent to improve dough performance.

GSH may be superior to other reducing agents. For example, sulfites are a commonly used reducing agent in the production of cookies and crackers but have limitations. Scheduled/coordinated use of GSH with an oxidizing agent could improve dough production by rapidly breaking down and reforming gluten linkages in dough and thereby adjusting dough extensibility.

Aspects of the disclosure relate to methods of making dough comprising providing a yeast cell comprising a Gsh1 variant described herein or a composition comprising such yeast cells or GSH purified from such yeast cell and preparing the dough using additional ingredients such as, e.g., flour, salt, oil, leavening, butter, sugar and/or eggs.

In some embodiments, provided is a method of baking bread using a high-speed process, including, e.g., bread made from frozen bread dough. In some embodiments, provided is a method of making dough with desirable extensibility, e.g., pizza dough, comprising providing a yeast cell comprising a Gsh1 variant described herein or a composition comprising such yeast cell or GSH purified from such yeast cell and preparing the dough.

Further provided is a method of making dough comprising providing a yeast cell comprising a Gsh1 variant described herein or a composition comprising such yeast cell or GSH purified from such yeast cell and an oxidizing agent and preparing the dough, e.g., to produce cookies and/or crackers. In some embodiments provided is a method of producing cookies and/or crackers comprising providing GSH isolated from a yeast cell comprising a Gsh1 variant described herein or a composition comprising such yeast cell or GSH purified from such yeast cell.

In some embodiments, provided is a method of bread making comprising a scheduled use of a composition comprising a yeast cell comprising a Gsh1 variant described herein and a composition comprising a yeast cell or GSH purified from such yeast cell and wild-type Gsh1 or a composition comprising wild-type Gsh1. In some embodiments, the method further comprises the use of an oxidizing agent. In some embodiments, the breadmaking method comprises the coordinated use of (i) the composition comprising a yeast cell comprising a Gsh1 variant or the composition comprising a yeast cell or GSH purified from such yeast cell and (ii) the wild-type Gsh1 or composition comprising the wild-type Gsh1 and, optionally, (iii) an oxidizing agent to break down and reform gluten linkages in the dough in a scheduled manner to expedite the breadmaking process.

### Fermented Beverages

Hydrogen sulfide (H₂S) is a volatile sulfur compound that is a naturally occurring byproduct of fermentation. It has a rotten egg smell that can negatively impact beer and wine. The main products of sulfur metabolism in yeast are H₂S, sulfur-containing amino acids, and GSH. Yeast engineered for high-production of GSH could be used for the production of fermented beverages as such strains divert sulfur away from hydrogen sulfide formation towards GSH, thus, selecting against these disulfide off-flavors. For example, incorporating the genetic changes described herein into existing brewing strains could reduce the maturation phase of the brewing process, which is commonly used to eliminate off-flavors. Accordingly, such strains can increase productivity and reduce cost of brewing.

Provided herein are methods of winemaking, comprising providing a yeast cell comprising a Gsh1 variant described herein or a composition comprising such yeast cell, wherein the high GSH content of such yeast cell diverts sulfur away from hydrogen sulfide formation, and selects against disulfide off-flavors, e.g., volatile sulfur compounds that are naturally occurring byproducts of fermentation.

### Nutraceuticals/Pharmaceuticals

GSH is one of the body's most potent antioxidants. Antioxidants are important for combating free radicals, which are highly reactive chemical species that damage critical biological molecules like DNA, proteins, carbohydrates, and lipids. Free radicals attack important macromolecules, leading to cell damage and homeostatic disruption. The antioxidant activity of GSH could confer widespread health benefits, including the prevention of diseases like cancer, liver disease, and Parkinsons, which are characterized by the presence of high levels of oxidants. Thus, strains of yeast described herein that produce high levels of GSH, or GSH purified from such yeast strains, could be used in nutraceuticals and/or pharmaceuticals to confer antioxidant activity.

Provided herein are methods of making nutraceuticals and/or pharmaceuticals comprising providing a yeast cell comprising a Gsh1 variant described herein or a composition comprising such a yeast cell or GSH purified from such a yeast cell to increase the intake of GSH in a subject. In some embodiments, a method comprises providing a nutraceutical and/or pharmaceutical to a subject to scavenge free radicals in the subject and reduce free radical damage to macromolecules including DNA, proteins, carbohydrates and lipids in the subject. In some embodiments, provided is a method comprising providing a nutraceutical and/or pharmaceutical to a subject having a disease or disorder including, but not limited to, a proliferative disease, e.g., cancer, or a degenerative disease, e.g., liver disease or Parkinson's disease. In some embodiments provided is a method of making a nutraceutical and/or pharmaceutical comprising providing GSH isolated from a yeast cell comprising a Gsh1 variant described herein or a composition comprising such isolated GSH.

### Cosmetics

Glutathione can protect skin against oxidative damage. In particular, it could protect against damage that leads to wrinkles, making GSH a useful form of anti-aging skincare. In addition, GSH also protects the skin against hyperpigmentation (e.g., age spots or sun spots), by inhibiting the production of the skin pigment melanin. Thus, strains of yeast described herein that produce high levels of GSH, or GSH purified from such yeast strains, could be used in cosmetics, including anti-aging and anti-hyperpigmentation cosmetics.

Further provided are methods of making a cosmetic comprising providing a yeast cell comprising a Gsh1 variant described herein or a composition comprising such yeast cell or GSH purified from such yeast cell, optionally, together with additional skin care ingredients to make a cosmetic. In some embodiments, a method comprises providing a cosmetic to the skin of a subject to scavenge free radicals in the skin and reduce free radical damage to macromolecules including DNA, proteins, carbohydrates and lipids of the skin of the subject. In some embodiments provided is a method of making a cosmetic comprising providing GSH isolated from a yeast cell comprising a Gsh1 variant described herein or a composition comprising such isolated GSH.

### Plant Protectants

The role of microbial-based biopesticides and biofertilizers in agricultural practices world-wide is expected to increase to implement more sustainable agriculture policies. Yeast strains producing high levels of GSH have the capacity to augment plant productivity by enhancing crop nutrition through their antioxidant properties. Thus, strains of yeast described herein that produce high levels of GSH, and extracts thereof, could be used as plant protectants during plant growth or after harvest.

Provided herein are methods of making a plant protectant comprising providing a yeast cell comprising a Gsh1 variant described herein or a composition comprising such a yeast cell or GSH purified from such a yeast cell and, optionally, additional ingredients to produce a plant protectant composition. In some embodiments provided is a method of making a plant protectant comprising providing GSH isolated from a yeast cell comprising a Gsh1 variant described herein or a composition comprising such isolated GSH.

### Animal Feed

A diet high in antioxidants is predicted to reduce the risk of many diseases. Antioxidants scavenge free radicals from the body's cells and prevent or reduce the damage caused by oxidation. Selenium, for example, is a common animal feed supplement with strong antioxidant and anti-inflammatory properties. GSH purified from yeast as described herein could be used to supplement or enhance the effects of selenium in animal feed. Yeast with high GSH content can also be used directly for animal feed.

Provided herein are methods of making an animal feed comprising providing a yeast cell comprising a Gsh1 variant described herein or a composition comprising such a yeast cell or GSH purified from such a yeast cell and, optionally, additional ingredients to produce an animal feed. In some embodiments provided is a method of making an animal feed comprising providing GSH isolated from a yeast cell comprising a Gsh1 variant described herein or a composition comprising such isolated GSH.

The phraseology and terminology used in this application is for the purpose of description and should not be regarded as limiting. The use of terms such as “including,” “comprising,” “having,” “containing,” “involving,” and/or variations thereof in this application, is meant to encompass the items listed thereafter and equivalents thereof as well as additional items.

The present invention is further illustrated by the following Examples, which should not be construed as limiting. The entire contents of the references (including literature references, issued patents, published patent applications, and co-pending patent applications) cited throughout this application are hereby expressly incorporated by reference.

### EXAMPLES

### Example 1. Identification of Feedback-Resistant (FBR) Alleles of GSH1

High-titer production of glutathione by yeast fermentation is limited by feedback inhibition of the first step in the biosynthetic pathway catalyzed by Gsh1. Expression of enzymatically active enzymes that are resistant to feedback inhibition by glutathione could yield strains with higher glutathione titers.

It was investigated whether Gsh1 variants could be identified that have increased production of the antioxidant peptide glutathione. Editing of the GSH1 gene was performed using multiplexed precision genome editing (Roy et al. (2018) Nature Biotechnol. 36: 512-520), which allows for generating and testing large genome editing libraries. A MAGESTIC site saturation editing library was designed for the GSH1 gene in a haploid laboratory strain. This library interrogated each codon that codes for the amino acids in the Gsh1 protein. Each amino acid change was encoded with two separate donors, such that there were two opportunities to generate and subsequently test every amino acid change (described more below).

The edited strains were generated and collected in 11 variant pools, which collectively represented 98.6% of all possible single amino acid variants. On average, each pool contained ~2,500 unique strains, whereby each strain harbored a genetic modification resulting in a single amino acid change in the Gsh1 protein. In addition to these non-synonymous genetic changes, each strain also contained a set of synonymous changes to the wild-type GSH1 sequence that were immediately adjacent to (either upstream or downstream of) the coding change. These synonymous changes (which have no impact on the amino acid sequence of Gsh1) were included to prevent recognition by the guide RNA during editing and subsequent cutting by Cas9; however, “upstream” and “downstream” variants of the same coding mutation have utility as internal controls.

This library of Gsh1 variants was subjected to competitive selection in various chemical stressors related to cellular glutathione levels, followed by next generation Illumina sequencing to count the frequency of each edited strain in the population.

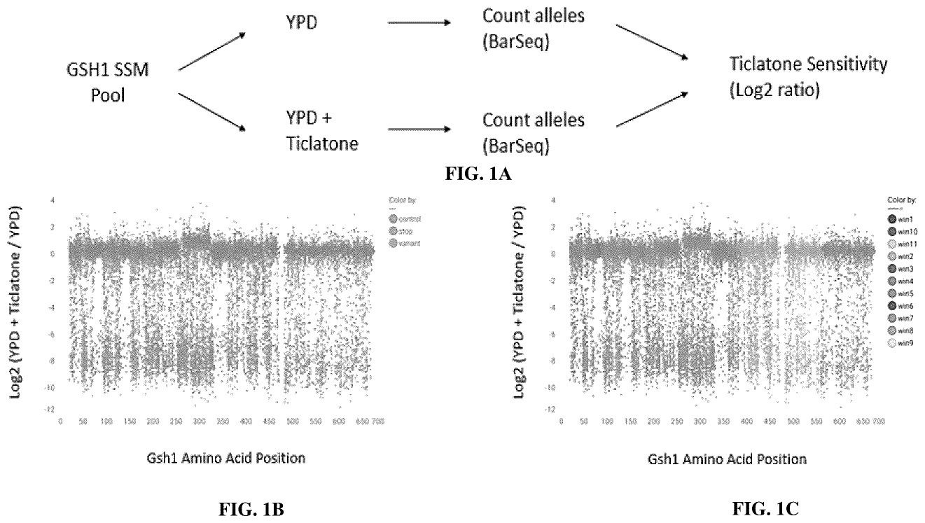

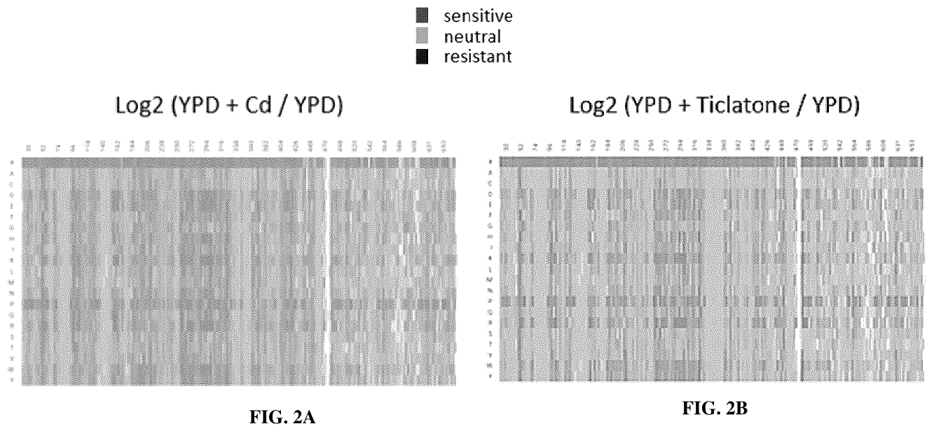

Pools of variant strains were cultured under various conditions to assess the performance of individual strains within each pool. To assess the impact of mutations on Gsh1 function, pools were cultured in cadmium chloride or ticlatone, two chemical toxins to which the Gsh1 protein confers resistance (i.e. in the absence of a functional Gsh1, yeast fitness is reduced in the presence of either toxin) (FIGS. 1 and 2). In the presence of either of these two toxins, Gsh1 is required to maintain cellular fitness. Therefore, loss-of-function mutations in Gsh1 can be readily identified based on sensitivity to these toxins.

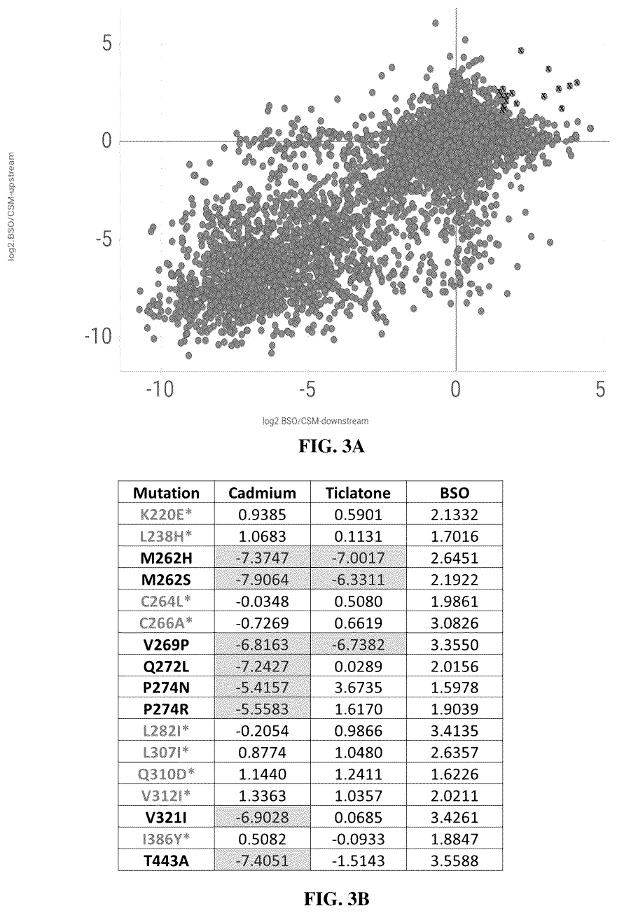

In addition, to assess the impact of mutations on feedback inhibition by GSH the library was grown in the presence of buthionine sulfoximine (BSO), a competitive inhibitor of Gsh1 that binds to many of the same amino acid residues in the Gsh1 protein that are bound by GSH (FIG. 3). Thus, amino acid changes that confer resistance to BSO are predicted to also confer resistance to GSH feedback inhibition.

A high-throughput screen was implemented to identify mutations in Gsh1 that conferred resistance to feedback inhibition by GSH but maintained high enzymatic activity. Mutations that reduced affinity with GSH without detrimentally affecting interaction between the substrate and enzymatic pocket were identified. The fitness of individual strains under the described conditions was assessed using Illumina sequencing to count the frequency of the edited GSH1 DNA in each condition. This is akin to DNA barcode sequencing and relies on the premise that the amount edited DNA is a direct reflection the abundance of that strain in the pool. Following growth, yeast cells from each of the 11 pools were collected by centrifugation and genomic DNA was extracted. PCR was used to amplify regions containing the edited DNA sequences (each pool representing a different region in GSH1). The primers for these PCR reactions contained Illumina adapters needed for sequencing. Raw read counts for each mutation were extracted from the resulting sequencing (fastq) files. These counts were normalized such that the total number of reads assigned to each pool was the same. A pseudocount of 1 was also added to each mutant count. Log 2 ratios were then calculated by dividing normalized read counts in a condition of interest by the normalized read counts in the control condition, and then taking the log base 2 of the result. Therefore, the log 2 ratios are a measure of the sensitivity or resistance conferred by a mutation of interest to a condition of interest.

Table 1 contains BSO resistant and active Gsh1 variants that were identified using the following log 2 score criteria: a) BSO Resistance (upstream) 22 1.5 AND Cd Resistance (upstream)>−2 AND Tic Resistance (upstream)>−2; or b) BSO Resistance (downstream)>1.5 AND Cd Resistance (downstream)>−2 AND Tic Resistance (downstream)>−2. For clarity, for Table 1, Gsh1 variants were considered BSO resistant and active if either the upstream or downstream variant met these criteria.

Amino acid substitution mutations were identified that were found to confer resistance to BSO and maintained activity (as determined by resistance to cadmium chloride and ticlatone) (Table 1 and FIG. 3). The mutations listed in Table 1 reflect those where either upstream or downstream versions were identified as conferring a high level of resistance to BSO, cadmium, and ticlatone, as these represent mutations that are resistant to feedback inhibition while maintaining normal activity.

Two cysteine mutations at amino acid position 266 in Gsh1 have previously been described as producing feedback resistance (C266A and C266S) (Biterova et al. J Biol Chem. 2010 May 7; 285(19): 14459-14466). C266A was also among the single mutations and top-performing combinatorial mutations identified in the screen described herein.

Various combinations between 10 of these mutations were introduced into the native yeast GSH1 gene in a laboratory strain background and evaluated for BSO-resistance. The selection of these 10 mutations was based on several criteria including high resistance scores to BSO, Ticlatone, and Cadmium, as well as good agreement between upstream and downstream strains for each mutation. In addition, the 10 mutations were sufficiently close enough to each other in the primary amino acid sequence to enable mutations to be encoded on a single synthesized oligonucleotide, 141 combinatorial variants exhibited BSO resistance exceeding those of single mutants while maintaining activity (Table 3). Twenty of the top-performing combinatorial variants were then introduced (by genomic integration) into a genetically engineered strain for glutathione production (YR040) and assessed by MTP and shake-flask fermentation followed by HPLC analysis. Several strains exhibited significant improvements in GSH production (Table 4).

TABLE 1
Amino acid positions and substitutions identified in Gsh1 variant
strains exhibiting enzymatic activity and BSO resistance

| Amino acid position | |
|---|---|
| (relative to SEQ ID NO: 1) | Amino acid substitution |
| 24 | D24Q |
| 26 | G26S |
| 28 | E28V |
| 30 | L30F |
| 30 | L30V |
| 32 | Y32M |
| 33 | I33A |
| 34 | F34M |
| 35 | Q35L |
| 36 | A36I |
| 37 | A37W |
| 38 | G38D |
| 39 | K39E |
| 39 | K39L |
| 39 | K39T |
| 40 | R40N |
| 41 | D41E |
| 42 | N42V |
| 42 | N42Y |
| 44 | P44V |
| 45 | L45F |
| 46 | F46C |
| 46 | F46E |
| 53 | Y53M |
| 55 | V55Y |
| 59 | D59E |
| 59 | D59L |
| 60 | D60K |
| 71 | H71C |
| 78 | L78G |
| 79 | N79G |
| 87 | E87M |
| 90 | D90S |
| 102 | L102F |
| 107 | A107D |
| 108 | S108Q |
| 108 | S108K |
| 108 | S108C |
| 117 | Y117T |
| 117 | Y117L |
| 117 | Y117Q |
| 118 | V118I |
| 121 | N121S |
| 123 | Q123M |
| 135 | E135P |
| 136 | Y136W |
| 136 | Y136C |
| 136 | Y136V |
| 136 | Y136F |
| 137 | A137T |
| 137 | A137F |
| 137 | A137V |
| 138 | R138V |
| 138 | R138T |
| 138 | R138N |
| 139 | Q139K |
| 142 | K142E |
| 142 | K142Q |
| 144 | N144T |
| 145 | L145P |
| 146 | H146A |
| 147 | V147T |
| 151 | S151P |
| 161 | R161L |
| 161 | R161V |
| 165 | P165H |
| 165 | P165T |
| 172 | D172Y |
| 174 | W174H |
| 176 | H176R |
| 176 | H176Y |
| 177 | K177H |
| 185 | F185Y |
| 198 | P198E |
| 199 | N199T |
| 202 | A202H |
| 203 | S203L |
| 206 | T206F |
| 206 | T206R |
| 210 | E210S |
| 220 | K220E |
| 220 | K220T |
| 220 | K220S |
| 220 | K220L |
| 220 | K220N |
| 225 | P225S |
| 229 | D229W |
| 235 | D235K |
| 235 | D235V |
| 236 | W236E |
| 238 | L238H |
| 238 | L238I |
| 239 | P239K |
| 240 | E240F |
| 242 | K242A |
| 242 | K242W |
| 243 | E243L |
| 245 | K245S |
| 246 | L246P |
| 246 | L246K |
| 247 | A247S |
| 248 | S248F |
| 248 | S248L |
| 249 | K249M |
| 249 | K249C |
| 250 | P250F |
| 250 | P250Y |
| 250 | P250Q |
| 250 | P250N |
| 251 | G251W |
| 252 | F252K |
| 253 | I253V |
| 264 | C264L |
| 264 | C264G |
| 264 | C264T |
| 264 | C264M |
| 264 | C264N |
| 265 | S265L |
| 265 | S265C |
| 265 | S265T |
| 266 | C266A |
| 266 | C266S |
| 269 | V269H |
| 269 | V269C |
| 269 | V269A |
| 271 | F271V |
| 276 | I276T |
| 276 | I276F |
| 276 | I276A |
| 277 | N277A |
| 277 | N277D |
| 277 | N277K |
| 277 | N277Q |
| 277 | N277I |
| 278 | K278Q |
| 278 | K278A |
| 281 | Y281E |
| 281 | Y281A |
| 281 | Y281K |
| 282 | L282I |
| 282 | L282C |
| 283 | Y283H |
| 285 | A285N |
| 286 | L286M |
| 287 | V287A |
| 288 | N288T |
| 288 | N288L |
| 288 | N288I |
| 289 | F289L |
| 289 | F289Y |
| 290 | A290T |
| 294 | L294A |
| 295 | A295V |
| 296 | F296Q |
| 296 | F296G |
| 296 | F296T |
| 297 | S297T |
| 300 | A300C |
| 301 | P301V |
| 302 | A302N |
| 302 | A302V |
| 302 | A302F |
| 302 | A302C |
| 303 | F303Y |
| 304 | K304R |
| 306 | W306T |
| 306 | W306L |
| 306 | W306Y |
| 306 | W306M |
| 307 | L307I |
| 307 | L307V |
| 309 | D309G |
| 309 | D309Q |
| 309 | D309N |
| 310 | Q310D |
| 310 | Q310T |
| 310 | Q310N |
| 310 | Q310L |
| 310 | Q310S |
| 312 | V312I |
| 314 | W314F |
| 315 | N315I |
| 315 | N315K |
| 316 | V316C |
| 316 | V316T |
| 316 | V316M |
| 317 | I317L |
| 317 | I317T |
| 317 | I317V |
| 318 | S318M |
| 318 | S318L |
| 318 | S318N |
| 318 | S318G |
| 318 | S318C |
| 318 | S318R |
| 319 | G319R |
| 319 | G319M |
| 319 | G319T |
| 319 | G319K |
| 321 | V321T |
| 323 | D323C |
| 330 | G330A |
| 332 | A332Y |
| 332 | A332C |
| 335 | L335D |
| 339 | N339M |
| 340 | K340G |
| 341 | N341C |
| 348 | K348V |
| 349 | D349C |
| 349 | D349I |
| 352 | D352W |
| 352 | D352V |
| 354 | V354T |
| 358 | P358H |
| 363 | S363A |
| 364 | S364M |
| 365 | V365A |
| 366 | D366I |
| 375 | F375Y |
| 376 | N376H |
| 376 | N376Q |
| 378 | T378G |
| 379 | Y379L |
| 381 | D381N |
| 383 | N383F |
| 385 | P385C |
| 386 | I386Y |
| 386 | I386T |
| 386 | I386S |
| 388 | E388F |
| 388 | E388A |
| 391 | L391C |
| 395 | L395A |
| 395 | L395Y |
| 398 | D398P |
| 400 | A400R |
| 401 | P401L |
| 404 | Y404L |
| 404 | Y404H |
| 406 | L406M |
| 410 | F410L |
| 410 | F410V |
| 413 | L413M |
| 414 | Y414C |
| 414 | Y414A |
| 422 | F422I |
| 429 | D429K |
| 430 | N430H |
| 431 | K431V |
| 431 | K431N |
| 432 | T432A |
| 432 | T432W |
| 434 | S434C |
| 436 | H436Q |
| 443 | T443S |
| 450 | F450I |
| 452 | P452I |
| 454 | T454W |
| 455 | Q455K |
| 455 | Q455H |
| 456 | Q456D |
| 457 | A457N |
| 458 | T458K |
| 460 | D460G |
| 461 | K461A |
| 461 | K461N |
| 462 | K462P |
| 464 | S464V |
| 465 | P465T |
| 465 | P465S |
| 469 | V469L |
| 491 | Y491L |
| 495 | D495E |
| 497 | I497Q |
| 497 | I497Y |
| 498 | L498I |
| 499 | T499V |
| 500 | F500K |
| 500 | F500E |
| 501 | S501R |
| 504 | I504N |
| 504 | I504Y |
| 514 | W514F |
| 514 | W514T |
| 514 | W514G |
| 514 | W514I |
| 515 | E515H |
| 515 | E515F |
| 515 | E515G |
| 517 | M517N |
| 518 | K518L |
| 519 | I519H |
| 527 | L527E |
| 528 | F528N |
| 528 | F528I |
| 529 | E529L |
| 535 | K535C |
| 536 | S536H |
| 536 | S536G |
| 538 | R538E |
| 540 | D540H |
| 542 | D542R |
| 544 | E544Q |
| 544 | E544D |
| 545 | T545I |
| 545 | T545S |
| 547 | D547V |
| 547 | D547M |
| 548 | Y548R |
| 548 | Y548Q |
| 548 | Y548S |
| 549 | S549F |
| 550 | I550G |
| 551 | S551I |
| 552 | E552R |
| 556 | N556H |
| 556 | N556M |
| 557 | P557M |
| 563 | P563M |
| 564 | Q564C |
| 567 | T567G |
| 568 | P568E |
| 571 | C571T |
| 576 | V576C |
| 579 | D579C |
| 581 | K581L |
| 581 | K581Q |
| 584 | K584F |
| 585 | H585M |
| 585 | H585T |
| 586 | S586Q |
| 587 | S587G |
| 587 | S587D |
| 587 | S587F |
| 588 | K588E |
| 588 | K588T |
| 589 | H589A |
| 590 | E590P |
| 594 | Y594S |
| 594 | Y594C |
| 597 | K597S |
| 600 | S600Y |
| 600 | S600H |
| 603 | A603C |
| 604 | S604H |
| 604 | S604N |
| 608 | P608F |
| 609 | T609V |
| 612 | K612C |
| 616 | N616I |
| 616 | N616V |
| 617 | F617C |
| 618 | V618L |
| 619 | L619W |
| 631 | S631I |
| 631 | S631F |
| 633 | S633M |
| 633 | S633R |
| 633 | S633W |
| 635 | N635S |
| 637 | D637F |
| 640 | S640I |
| 641 | T641A |
| 642 | C642G |
| 643 | D643F |
| 643 | D643Y |
| 643 | D643M |
| 655 | L655I |
| 656 | T656I |
| 657 | S657N |
| 664 | A664F |
| 665 | E665H |
| 668 | K668I |
| 668 | K668W |
| 669 | K669D |
| 670 | N670I |

Table 2 contains Gsh1 variants that were considered BSO resistant and active (upstream and downstream) when BSO Resistance (upstream AND downstream)>1, AND Cd Resistance (upstream AND downstream)>−2, AND Tic Resistance (upstream AND downstream)>−2. AVG BSO in Table 2 means average of [log 2.BSO/CSM-downstream] and [log 2.BSO/CSM-upstream].

TABLE 2
Subset of BSO resistant and active variants

| Amino | | log2.Cd/ | log2.Tic/ | log2.BSO/ | log2.Cd/ | log2.Tic/ | log2.BSO/ | |
|---|---|---|---|---|---|---|---|---|
| acid | | YPD- | YPD- | CSM- | YPD- | YPD- | CSM- | |
| position | Mutation | upstream | upstream | upstream | downstream | downstream | downstream | AVG BSO |
| 282 | L282I | −0.2092 | 1.19743 | 3.70729 | −0.2016 | 0.77572 | 3.11963 | 3.413460265 |
| 266 | C266A | −0.4076 | 1.39432 | 2.69864 | −1.0461 | −0.0705 | 3.46648 | 3.082559327 |
| 307 | L307I | 0.83178 | 0.91532 | 2.29814 | 0.92309 | 1.18066 | 2.97335 | 2.635744137 |
| 137 | A137T | −0.0244 | −0.4895 | 1.03854 | 0.21193 | −0.2123 | 4.10876 | 2.57365066 |
| 220 | K220E | 0.8361 | 0.53761 | 2.70277 | 1.04094 | 0.64265 | 1.56369 | 2.133228816 |
| 312 | V312I | 1.47958 | 0.93076 | 2.35214 | 1.19301 | 1.14054 | 1.69009 | 2.021117364 |
| 404 | Y404L | 0.98174 | −0.0323 | 1.10688 | 0.63581 | −0.9376 | 2.90702 | 2.00695186 |
| 314 | W314F | −0.692 | 0.92397 | 2.70628 | −1.4659 | 0.42883 | 1.27659 | 1.991437214 |
| 264 | C264L | 0.56822 | 0.88551 | 2.53425 | −0.6379 | 0.13042 | 1.43788 | 1.986061474 |
| 436 | H436Q | −0.5584 | 0.46813 | 2.71712 | 0.20212 | 0.00346 | 1.12281 | 1.91996814 |
| 386 | I386Y | 0.92974 | 0.01601 | 2.10433 | 0.08659 | −0.2027 | 1.6651 | 1.884717066 |
| 317 | I317L | 0.40697 | 1.15412 | 2.18794 | −0.3092 | 1.05378 | 1.38175 | 1.784846498 |
| 545 | T545I | 0.62574 | 0.82084 | 1.33062 | 0.35504 | −0.6549 | 2.19539 | 1.763005266 |
| 637 | D637F | −1.6076 | −0.7062 | 1.11739 | 1.14858 | −0.333 | 2.34607 | 1.731733121 |
| 317 | I317T | 0.34586 | 0.83546 | 1.09034 | 1.06835 | 0.8321 | 2.35686 | 1.723597867 |
| 287 | V287A | 0.78562 | 0.47025 | 2.32829 | 0.91532 | 1.09624 | 1.1156 | 1.721948378 |
| 265 | S265L | −0.2615 | 1.44414 | 1.26185 | −0.0836 | 0.98269 | 2.1502 | 1.706022635 |
| 238 | L238H | 1.60953 | 0.17189 | 1.81406 | 0.52705 | 0.05435 | 1.5891 | 1.701578278 |
| 461 | K461A | 0.71222 | 0.71213 | 2.26998 | 0.71519 | 0.32328 | 1.05713 | 1.663553238 |
| 306 | W306T | 0.85605 | 0.90673 | 2.2199 | −0.5131 | 1.08496 | 1.10144 | 1.660672672 |
| 307 | L307V | 1.68177 | 1.62049 | 2.30969 | 2.32558 | 3.06932 | 1.00937 | 1.659530797 |
| 452 | P452I | −1.5025 | 0.60898 | 1.45426 | −0.9157 | 1.16752 | 1.79999 | 1.627125684 |
| 277 | N277A | 1.46814 | 0.54677 | 1.07562 | 1.73337 | 1.68183 | 2.17414 | 1.624881149 |
| 310 | Q310D | 1.36786 | 2.04982 | 1.57451 | 0.92005 | 0.43231 | 1.67065 | 1.622579443 |
| 318 | S318M | 1.78947 | 1.93654 | 2.01544 | 1.65858 | 1.96128 | 1.07303 | 1.544237616 |
| 137 | A137F | 1.47455 | 0.30941 | 1.9133 | 0.30532 | 0.31029 | 1.09561 | 1.504456161 |
| 264 | C264G | −1.2192 | 1.18533 | 1.38542 | −0.8743 | 0.96041 | 1.5375 | 1.461458323 |
| 318 | S318L | 0.18269 | 0.94516 | 1.50169 | 1.06188 | 1.06582 | 1.4078 | 1.454747182 |
| 608 | P608F | 0.10236 | −0.1631 | 1.24853 | 1.36556 | 0.2914 | 1.63754 | 1.443033069 |
| 151 | S151P | 0.28719 | −0.0703 | 1.57184 | −0.2091 | 0.11335 | 1.25879 | 1.415316815 |
| 238 | L238I | 1.50754 | 0.78042 | 1.60262 | 0.4611 | 0.37421 | 1.21789 | 1.410255541 |
| 315 | N315I | 1.64174 | 1.8547 | 1.29191 | 0.10548 | −0.3811 | 1.52711 | 1.409508296 |
| 315 | N315T | 0.81944 | 1.71874 | 1.28255 | 1.57911 | 1.65073 | 1.46161 | 1.372081449 |
| 246 | L246P | 0.99922 | 0.07969 | 1.53662 | 1.33701 | 0.72261 | 1.18645 | 1.36153915 |
| 59 | D59S | −0.2543 | 0.83321 | 1.48787 | −0.6772 | −0.2388 | 1.13091 | 1.309388514 |
| 568 | P568A | −0.0093 | 0.30666 | 1.20662 | 1.49023 | 1.52872 | 1.38893 | 1.297775033 |
| 149 | S149Q | 0.31986 | 0.36834 | 1.26201 | 0.99599 | 0.43687 | 1.28088 | 1.271447325 |
| 310 | Q310I | 2.21279 | 1.11359 | 1.29121 | 0.8819 | 2.12985 | 1.23213 | 1.261669308 |
| 659 | L659V | 0.37521 | −0.0472 | 1.26294 | 0.90833 | −0.1104 | 1.17427 | 1.218603748 |
| 320 | A320C | 1.4925 | 0.81002 | 1.11653 | 1.07221 | −0.1177 | 1.26088 | 1.188705556 |
| 298 | A298G | −0.3718 | 0.97459 | 1.07496 | −0.8961 | 1.12857 | 1.29898 | 1.186970243 |
| 173 | P173F | 0.20387 | 0.0655 | 1.00668 | 1.68073 | 0.64894 | 1.32868 | 1.167679424 |
| 104 | A104V | −1.1018 | 0.38491 | 1.06235 | −1.0403 | 0.01461 | 1.24181 | 1.152082009 |
| 374 | F374I | −0.9494 | −0.0168 | 1.23888 | −0.9723 | −0.8681 | 1.02957 | 1.134223068 |
| 223 | A223F | 0.34858 | 0.23354 | 1.05485 | 0.397 | 0.03555 | 1.19507 | 1.124959155 |
| 315 | N315Q | 1.94693 | 0.82432 | 1.1976 | 0.88845 | 0.68305 | 1.04679 | 1.122198361 |
| 239 | P239Q | 0.80585 | −0.2647 | 1.04393 | 0.3095 | −0.5488 | 1.17706 | 1.110494407 |
| 227 | T227H | 0.58837 | 0.57753 | 1.05099 | 0.90507 | 0.58055 | 1.08844 | 1.069716365 |

TABLE 3
Gsh1 amino acid substitutions in combinatorial variant strains
Amino acid substitutions

C264L:C266A:Q310D:V312I
K220E:C264L:C266A:Q310D
L282I:Q310D:V312I
K220E:C264L:L282I:L307I
K220E:C266A:Q310D:G319R
L3071:V312I:G319R
L238H:C264L:Q310D:G319R
C266A:Q310D:V312I:G319R
C264L:C266A:L282I:Q310D
C264L:N277D:L282I:Q310D
K220E:C264L:C266A:L307I
C266A:N277D:Q310D:V312I
L238H:L282I:Q310D:V312I
C264L:C266A:L282I:L307I
K220E:L238H:C264L:G319R
C266A:N277D
K220E:L238H:V312I:G319R
K220E:C264L:C266A:V312I
L282I:Q310D:G319R
C266A:L307I:Q310D
K220E:C264L:Q310D
L238H:C264L:C266A:N277D
C266A:L282I:Q310D:G319R
K220E:C266A:N277D:L282I
K220E:G319R
K220E:L238H:Q310D:G319R
K220E:L238H:C266A:N277D
C266A:N277D:L282I
L238H:C266A:L282I:Q310D
L238H:C264L:C266A:L307I
L238H:C266A:Q310D:V312I
C264L:C266A:L282I:V312I
K220E:C264L:C266A
K220E:C264L:L307I
K220E:C264L:N277D:Q310D
L238H:C266A:Q310D:G319R
L238H:C264L:C266A:Q310D
K220E:N277D:L282I:Q310D
C264L:C266A:N277D:L307I
K220E:C266A:L282I:Q310D
N277D:L282I:Q310D:G319R
C264L:C266A:Q310D
L238H:C266A:N277D:L282I
K220E:C266A:L307I:V312I
L238H:C264L:C266A:L282I
C264L:C266A:N277D:Q310D
K220E:N277D:Q310D:V312I
C264L:N277D:Q310D:G319R
K220E:L238H:C266A:Q310D
L238H:Q310D:G319R
C264L:C266A:L307I:V312I
K220E:N277D:Q310D:G319R
K220E:L238H:L282I:V312I
C264L:L282I:G319R
K220E:L282I:G319R
C266A:L282I:G319R
K220E:C264L:L307I:V312I
K220E:C266A:V312I:G319R
K220E:L307I:V312I
K220E:N277D:L282I:G319R
K220E:C266A:N277D
C266A:N277D:L307I
C266A:N277D:L282I:V312I
C266A:Q310D:V312I
K220E:C266A:Q310D:V312I
K220E:C266A:L307I:G319R
C264L:C266A:N277D
C264L:C266A:L282I:G319R
L238H:C264L:G319R
C264L:N277D:V312I:G319R
L238H:C266A:L282I:V312I
K220E:C266A:L282I:L307I
C266A:L307I:V312I
C266A:L282I:V312I
K220E:C264L:C266A:G319R
C264L:C266A:V312I:G319R
L238H:C264L:C266A
C264L:N277D:Q310D
L238H:C264L:L282I
K220E:C266A
L238H:C266A:N277D
K220E:C266A:V312I
L238H:C266A:L307I
K220E:C266A:N277D:L307I
L238H:C266A:V312I
K220E:C266A:L282I
L238H:C266A:Q310D
K220E:L238H:C264L:V312I
C266A:G319R
K220E:C266A:L307I
L238H:N277D:V312I:G319R
L238H:Q310D
C266A:L282I:L307I
L238H:C266A:L282I:G319R
C266A:Q310D:G319R
L238H:N277D:Q310D
L238H:C264L:N277D:G319R
C266A:L282I
K220E:C266A:L282I:G319R
C266A:N277D:Q310D:G319R
K220E:L238H:Q310D
C266A:N277D:G319R
L238H:L2821:L307I:G319R
L238H:C266A
C264L
K220E:C266A:G319R
L238H:N277D:L282I:G319R
L238H:C264L:C266A:G319R
C266A:N277D:Q310D
L238H:C266A:N277D:V312I
C266A:L307I
C264L:C266A:N277D:V312I
C266A:N277D:L307I:V312I
C264L:C266A
C264L:C266A:G319R
L238H:C266A:N277D:L307I
K220E:L238H:L3071:G319R
L238H:C266A:G319R
K220E:N277D:V312I:G319R
V312I:G319R
K220E:C266A:N277D:G319R
K220E:C264L
L238H:C266A:L282I:L307I
L238H:L282I:Q310D
C264L:N277D:V312I
K220E:L238H:C266A:V312I
C264L:C266A:L307I
L238H:C266A:N277D:G319R
K220E:L238H:C266A
C266A:Q310D
L238H:C266A:L282I
L238H:C266A:L307I:G319R
C266A:L307I:G319R
K220E:N277D:G319R
C266A:N277D:V312I
L238H:V312I
L238H:C264L:C266A:V312I
C264L:C266A:L307I:G319R
K220E:C264L:V312I:G319R
N277D:L282I:L307I:G319R
L238H:N277D:L282I:V312I

TABLE 4
GSH1 combinatorial variant strains exhibiting significant
improvement in glutathione production in shake-flask.

| | | Avg GSH | StdDev GSH |
|---|---|---|---|
| Strain | Description | (g/100 g dw) | (g/100 g dw) |
| YR040 | wild-type | 20.05 | 0.62 |
| YR640 | YR040 + ChrXII-5::pTEF1-GSH1- | 28.20 | 2.31 |
| | variant7-C266A, N277D, V312I | | |
| YR642 | YR040 + ChrXII-5::pTEF1-GSH1- | 22.97 | 1.85 |
| | variant9-K220E, L238H, C266A | | |
| YR647 | YR040 + ChrXII-5::pTEF1-GSH1- | 27.47 | 1.12 |
| | variant14-K220E, L238H, C266A, | | |
| | V312I | | |
| YR649 | YR040 + ChrXII-5::pTEF1-GSH1- | 26.08 | 4.78 |
| | variant16-L238H, C264L, C266A, | | |
| | G319R | | |
| YR654 | YR040 + ChrXII-5::pTEF1-GSH1- | 12.86 | 1.49 |
| | wildtype | | |

### Example 2. Heterologous Expression of GCS-GS

A second approach for increasing GSH production was based on bioprospecting and identification of bacterial bifunctional enzymes that encode both steps of glutathione biosynthesis and exhibit innate resistance to feedback inhibition (Gopal et al., 2005, Janowiak and Griffith, 2005, Vergauwen et al., 2006). Specifically, the issue of GSH feedback inhibition was addressed by expressing either Streptococcus thermophilus (S. thermophilus) or Streptococcus agalactiae (S. agalactiae) GCS-GS enzyme in yeast. It was reasoned that heterologous expression of these bifunctional enzymes alone or in combination with GSH1 FBR mutations identified in Example 1, would increase the amount of GSH produced by yeast fermentation.

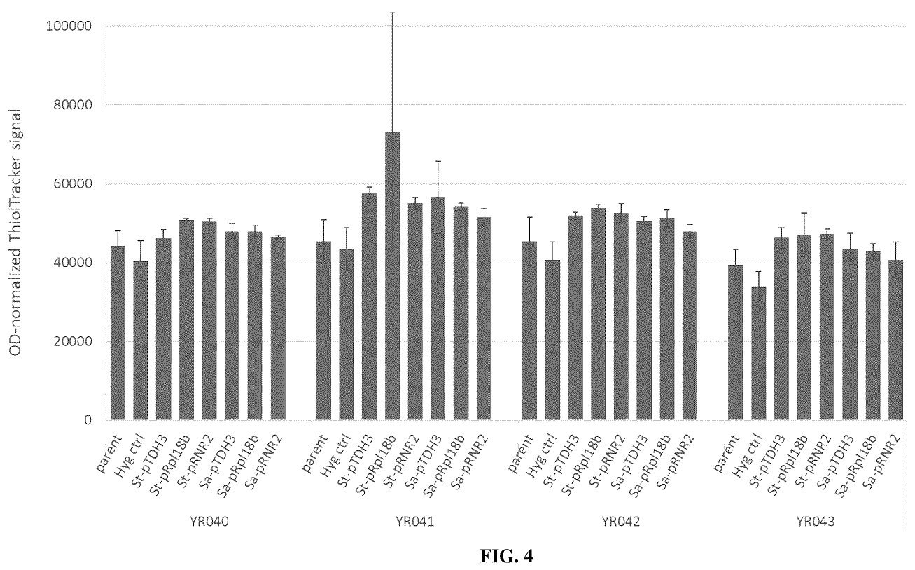

Commercial gene synthesis was used to generate codon-optimized (for yeast) bi-functional glutathione synthesis enzymes of bacterial origin (GCS-GS) encoding bifunctional enzymes from S. thermophilus and S. agalactiae. Heterologous expression of either enzyme using different strength promoters (e.g., TDH3, Rp118b, and RNR2) was found to increase glutathione production in multiple genetic backgrounds (e.g., YR040, YR041, YR042, YR043) as measured by a ThiolTracker fluorescence plate reader assay (FIG. 4).

The S. thermophilus GCS-GS enzyme was integrated into the diploid parent of an industrial strain (YR043). This strain is of the W303 background. Heterologous expression was found to increase glutathione titers following cultivation in shake-flask and GSH quantification via HPLC (FIG. 5), with the strongest effect observed with a TDH3 promoter.

### Example 3. Combining FBR GSH1 Alleles and GCS-GS

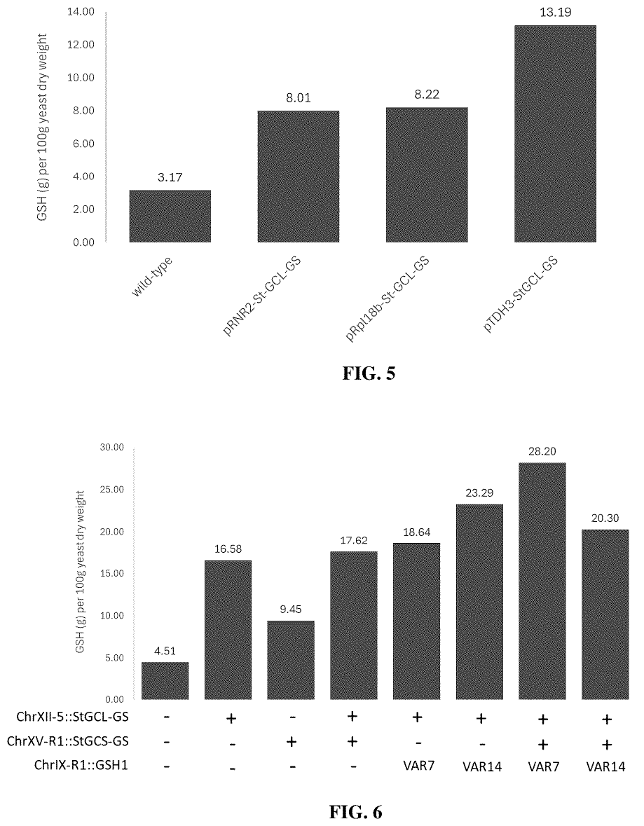

FBR alleles of GSH1 and expression cassettes for GCS-GS were introduced into the YR043 background. Specifically, two combinatorial FBR alleles (variant 7, comprising C266A, N277D and V312I, and variant 14, comprising K220E, L238H, C266A, and V312I, both listed in Table 4) and an expression cassette of StGCS-GS integrated at two different sites (ChrXV-R1 and ChrIX-R1) were used. Glutathione production of the resulting strain panel was then measured in a shake-flask (FIG. 6). Combining GSH1 FBR alleles with GCS-GS heterologous expression led to an additional increase in GSH production. Therefore, both approaches, expressing FBR alleles of GSH and expressing heterologous GCS-GS were found to produce strains with significantly higher glutathione titers in shake-flask, and even higher production was observed when the two approaches were combined.

### Example 4. Production in Bioreactors

Yeast cells as described herein will be produced in bioreactors and byproduct formation will be assessed. In addition, downstream purification of GSH will be performed.

### EQUIVALENTS

Those skilled in the art will recognize or be able to ascertain using no more than routine experimentation, many equivalents to the specific embodiments of the invention described in this application. Such equivalents are intended to be encompassed by the following claims.

All references, including patent documents, are incorporated by reference in their entirety.

1. A gamma glutamylcysteine synthetase comprising an amino acid sequence that is at least 90% identical to the sequence of SEQ ID NO: 1, wherein the amino acid sequence of the gamma glutamylcysteine synthetase comprises an amino acid substitution relative to the sequence of SEQ ID NO: 1 at one or more amino acid positions corresponding to positions K220, L238, C264, N277, V312, or G319 of SEQ ID NO: 1.

2. A gamma glutamylcysteine synthetase comprising an amino acid sequence that is at least 90% identical to the sequence of SEQ ID NO: 1, wherein the amino acid sequence of the gamma glutamylcysteine synthetase comprises an amino acid substitution relative to the sequence of SEQ ID NO: 1 at two or more amino acid positions listed in Table 1 or Table 2.

3. (canceled)

4. The gamma glutamylcysteine synthetase of claim 1, wherein the amino acid sequence of the gamma glutamylcysteine synthetase further comprises an amino acid substitution at a position corresponding to position C266 of SEQ ID NO: 1.

5. The gamma glutamylcysteine synthetase of claim 2, wherein the amino acid sequence of the gamma glutamylcysteine synthetase comprises an amino acid substitution at two or more positions corresponding to positions K220, L238, C264, C266, N277, V312, or G319 of SEQ ID NO: 1.

6. The gamma glutamylcysteine synthetase of claim 2, wherein the amino acid sequence of the gamma glutamylcysteine synthetase comprises one or more of the following amino acid substitutions relative to the sequence of SEQ ID NO: 1: K220E, L238H, C264L, C266A, N277D, V312I, or G319R.

7. The gamma glutamylcysteine synthetase of claim 2, wherein the amino acid sequence of the gamma glutamylcysteine synthetase comprises the following amino acid substitutions relative to the sequence of SEQ ID NO: 1:
C266A, N277D, and V312I;
K220E, L238H, and C266A;
K220E, L238H, C266A, and V312I; or
L238H, C264L, C266A, and G319R.

8. The gamma glutamylcysteine synthetase of claim 2, wherein the gamma glutamylcysteine synthetase is resistant to feedback inhibition by glutathione (GSH).

9. (canceled)

10. A yeast cell comprising the gamma glutamylcysteine synthetase of claim 2.

11. The yeast cell of claim 10, wherein the yeast cell is a S. cerevisiae cell.

12-15. (canceled)

16. The yeast cell of claim 10, further comprising a heterologous glutathione biosynthesis bifunctional protein.

17. The yeast cell of claim 16, wherein the heterologous glutathione biosynthesis bifunctional protein is a bacterial glutathione biosynthesis bifunctional protein.

18. The yeast cell of claim 17, wherein the bacterial glutathione biosynthesis bifunctional protein is a S. thermophilus or S. agalactiae glutathione biosynthesis bifunctional protein.

19. The yeast cell of claim 16, wherein the heterologous glutathione biosynthesis bifunctional protein is expressed in the yeast cell under the control of a TDH3 promoter, a Rp118b promoter, or a RNR2 promoter.

20. The yeast cell of claim 10, wherein the yeast cell exhibits increased GSH production relative to a control yeast cell.

21. A composition comprising the yeast cell of claim 10, wherein the composition comprises one or more of: yeast extract, active dry yeast, instant dry yeast, crumbled yeast, cream yeast, or baker's yeast.

22.-29. (canceled)

30. A method for increasing antioxidant content in a product comprising using a yeast cell of claim 10, wherein the product is a bread, a wine, a nutraceutical, a pharmaceutical, or a plant protectant.

31-32. (canceled)

33. The yeast cell of claim 16, wherein the glutathione biosynthesis bifunctional protein comprises an amino acid sequence that is at least 90% identical to the sequence of SEQ ID NO: 3 or SEQ ID NO: 5, or a conservatively substituted version thereof.

34. A yeast cell comprising:
(a) a heterologous glutathione biosynthesis bifunctional protein; and
(b) a gamma glutamylcysteine synthetase comprising an amino acid sequence that is at least 90% identical to the sequence of SEQ ID NO: 1, wherein the amino acid sequence of the gamma glutamylcysteine synthetase comprises an amino acid substitution relative to the sequence of SEQ ID NO: 1 at one or more amino acid positions listed in Table 1 or Table 2.

35. The yeast cell of claim 34, wherein the glutathione biosynthesis bifunctional protein comprises an amino acid sequence that is at least 90% identical to the sequence of SEQ ID NO: 3 or SEQ ID NO: 5, or a conservatively substituted version thereof.

36. A method for increasing antioxidant content of a product comprising using a yeast cell of claim 34, wherein the product is a bread, a wine, a nutraceutical, a pharmaceutical, or a plant protectant.


SEQUENCE LISTING (6 sequences)

SEQ ID NO: 1 - protein, 678 aa, Saccharomyces cerevisiae
MGLLALGTPLQWFESRTYNEHIRDEGIEQLLYIFQAAGKRDNDPLFWGDELEYMVVDFDDKERNSMLDVCHDKILTELNMEDSSLCEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 2 - DNA, 2037 bp, Saccharomyces cerevisiae
  source 1..2037: mol_type = genomic DNA
atgggactcttagctttgggcacgcctttgcagtggtttgagtctaggacgtacaatgaacacataagggatgaaggtatcgagcagttgttgtatattttccaagctgctggtaaaagagacaatgaccctcttttttggggagacgagcttgagtacatggttgtagattttgatgataaggagagaaattctatgctcgacgtttgccatgacaagatactcactgagcttaatatggaggattcgtccctttgtgaggctaacgatgtgagttttcaccctgagtatggccggtatatgttagaggcaacaccagcttctccatatttgaattacgtgggtagttacgttgaggttaacatgcaaaaaagacgtgccattgcagaatataagctatctgaatatgcgagacaagatagtaaaaataacttgcatgtgggctccaggtctgtccctttgacgctgactgtcttcccgaggatgggatgccccgactttattaacattaaggatccgtggaatcataaaaatgccgcttccaggtctctgtttttacccgatgaagtcattaacagacatgtcaggtttcctaacttgacagcatccatcaggaccaggcgtggtgaaaaagtttgcatgaatgttcccatgtataaagatatagctactccagaaacggatgactccatctacgatcgagattggtttttaccagaagacaaagaggcgaaactggcttccaaaccgggtttcatttatatggattccatgggttttggcatgggctgttcgtgcttacaagtgacctttcaggcacccaatatcaacaaggcacgttacctgtacgatgcattagtgaattttgcacctataatgctagccttctctgccgctgcgcctgcttttaaaggttggctagccgaccaagatgttcgttggaatgtgatatctggtgcggtggacgaccgtactccgaaggaaagaggtgttgcgccattactacccaaatacaacaagaacggatttggaggcattgccaaagacgtacaagataaagtccttgaaataccaaagtcaagatatagttcggttgatcttttcttgggtgggtcgaaatttttcaataggacttataacgacacaaatgtacctattaatgaaaaagtattaggacgactactagagaatgataaggcgccactggactatgatcttgctaaacattttgcgcatctctacataagagatccagtatctacattcgaagaactgttgaatcaggacaacaaaacgtcttcaaatcactttgaaaacatccaaagtacaaattggcagacattacgttttaaaccccccacacaacaagcaaccccggacaaaaaggattctcctggttggagagtggaattcagaccatttgaagtgcaactattagattttgagaacgctgcgtattccgtgctcatatacttgattgtcgatagcattttgaccttttccgataatattaacgcatatattcatatgtccaaagtatgggaaaatatgaagatagcccatcacagagatgctatcctatttgaaaaatttcattggaaaaaatcatttcgcaacgacaccgatgtggaaactgaagattattctataagcgagattttccataatccagagaatggtatatttcctcaatttgttacgccaatcctatgccaaaaagggtttgtaaccaaagattggaaagaattaaagcattcttccaaacacgagagactatactattatttaaagctaatttctgatagagcaagcggtgaattgccaacaacagcaaaattctttagaaattttgtactacaacatccagattacaaacatgattcaaaaatttcaaagtcgatcaattatgatttgctttctacgtgtgatagacttacccatttagacgattcaaaaggtgaattgacatcctttttaggagctgaaattgcagaatatgtaaaaaaaaataagccttcaatagaaagcaaatgttaa

SEQ ID NO: 3 - protein, 754 aa, Streptococcus thermophilus
MTLNQLLQKLEATSPILQANFGIERESLRVDRQGQLVHTPHPSCLGARSFHPYIQTDFCEFQMELITPVAKSTTEARRFLGAITDVAGRSIATDEVLWPLSMPPRLKAEEIQVAQLENDFERHYRNYLAEKYGTKLQAISGIHYNMELGKDLVEALFQESDQTDMIAFKNALYLKLAQNYLRYRWVITYLFGASPIAEQGFFDQEVPEPVRSFRNSDHGYVNKEEIQVSFVSLEDYVSAIETYIEQGDLNAEKEFYSAVRFRGQKVNRSFLDKGITYLEFRNFDLNPFERIGISQTTMDTVHLLILAFLWLDSPENVDQALAQGHALNEKIALSHPLKPLPSEAKTQDIVTALDQLVQHFGLGDYHQDLVKQVKAAFADPNQTLSAQLLPYIKDKSLAEFALNKALAYHDYDWTAHYALKGYEEMELSTQMLLFDAIQKGIHFEILDEQDQFLKLWHQDHVEYVKNGNMTSKDNYVVPLAMANKTVTKKILADAGFSVPSGDEFTSLEEGLAYYPLIKDKQIVVKPKSTNFGLGISIFQEPASLDNYQKALEIAFAEDTSVLVEEFIPGTEYRFFILDGRCEAVLLRVAANVIGDGKHTIRELVAQKNANPLRGRDHRSPLEIIELGDIEQLMLAQQGYTPDDILPEGKKVNLRRNSNISTGGDSIDVTETMDSSYQELAAAMATSMGAWACGVDLIIPDETQIATKENPHCTCIELNFNPSMYMHTYCAEGPGQAITTKILDKLFPEIVAGQT

SEQ ID NO: 4 - DNA, 2262 bp, synthetic construct
  source 1..2262: mol_type = other DNA
atgactttgaaccaattgttgcaaaagctggaagctacttctccaatcttacaggccaacttcggtattgaaagagaatctttacgtgttgacagacaaggtcaattggttcacaccccacacccatcctgtttgggtgccagatcattccacccatacatccaaactgacttctgtgaattccaaatggaattaatcactccagttgccaaatctaccacagaagctagaagatttttgggtgccattactgatgttgccggtagatccatagctactgatgaagtcttatggccactgtccatgccaccaagattgaaagctgaagaaatccaagtcgctcaattagaaaacgattttgaaagacattacagaaactatttggctgaaaagtacggtaccaagctacaagccatctccggtattcactacaatatggaattgggtaaagatttggtcgaagccttgttccaagaatccgaccaaaccgacatgattgctttcaagaacgccttgtacttgaagttggcccaaaactacttaagatacagatgggttatcacttatttgtttggtgcttctccaattgccgaacaaggtttctttgaccaagaagttccagaaccagtcagatcattcagaaattctgaccacggttacgttaataaggaagaaattcaagtttccttcgtttctctcgaagattacgtttctgccatcgaaacctatattgaacaaggcgatttgaacgccgaaaaagaattctactccgctgtcaggttcagaggtcaaaaggttaacagatctttcttggacaaaggcattacctacttggaattcagaaacttcgatcttaaccctttcgaaagaatcggtatctctcaaaccaccatggacactgtccacttgcttatcttagctttcttatggttggattctccagaaaacgtcgaccaagcattggctcaaggtcacgctttgaacgaaaagattgctttatctcatccattgaagccattgccgtctgaagctaagacccaagatattgtcaccgctttggaccaattggtccaacacttcggtttaggtgattaccatcaagacttggttaagcaagttaaggccgcctttgcagaccctaaccaaacgttatccgctcaactgttgccatacatcaaggataagtctttggctgaatttgctttgaacaaggctttggcttaccacgactacgactggactgctcactacgctttgaagggttacgaagaaatggaattgtccactcaaatgttgttgttcgacgctatccaaaagggtattcatttcgagatcttggatgaacaagatcaattcctcaagttgtggcaccaagatcacgttgaatacgtgaagaacggtaacatgacctccaaggacaattatgttgttccattagctatggctaacaagactgttaccaagaagatcttggctgacgctggtttctctgtcccatctggtgatgaattcacttctttggaagaaggtttggcttactacccattgattaaagacaagcaaatcgttgtcaagccaaagagcaccaacttcggtttgggtatttccatttttcaagaaccagcttctttggacaactaccaaaaggctttggaaatcgctttcgctgaagacacctcagttttggtcgaagaattcattccaggtactgaatacagattcttcatcctagacggtagatgtgaagccgttttgttgagagttgctgctaacgttatcggtgatggtaagcacactatccgtgaattggtcgctcaaaaaaatgctaacccattaagaggtcgtgatcaccgttccccattggaaattattgaattgggagacatagaacaattgatgttggctcaacaaggttacaccccagatgacatcttgccagaaggtaagaaggtcaacttgagaagaaactctaacatctctactggtggcgacagtattgatgtcaccgaaaccatggattcttcttaccaagagctagctgctgccatggctacctccatgggtgcttgggcttgtggtgttgacttgatcatcccagacgaaactcaaattgctaccaaggaaaaccctcattgcacctgtatcgaattgaacttcaacccttccatgtacatgcacacttactgtgctgaaggtccaggtcaagctattactactaagattttagacaagctattcccagaaatcgttgctggtcaaact

SEQ ID NO: 5 - protein, 750 aa, Streptococcus agalactiae
MIIDRLLQRSHSHLPILQATFGLERESLRIHQPTQRVAQTPHPKTLGSRNYHPYIQTDYSEPQLELITPIAKDSQEAIRFLKAISDVAGRSINHDEYLWPLSMPPKVREEDIQIAQLEDAFEYDYRKYLEKTYGKLIQSISGIHYNLGLGQELLTSLFELSQADNAIDFQNQLYMKLSQNFLRYRWLLTYLYGASPVAEEDFLDQKLNNPVRSLRNSHLGYVNHKDIRISYTSLKDYVNDLENAVKSGQLIAEKEFYSPVRLRGSKACRNYLEKGITYLEFRTFDLNPFSPIGITQETVDTVHLFLLALLWIDSSSHIDQDIKEANRLNDLIALSHPLEKLPNQAPVSDLVDAMQSVIQHFNLSPYYQDLLESVKRQIQSPELTVAGQLLEMIEGLSLETFGQRQGQIYHDYAWEAPYALKGYETMELSTQLLLFDVIQKGVNFEVLDEQDQFLKLWHNSHIEYVKNGNMTSKDNYIVPLAMANKVVTKKILDEKHFPTPFGDEFTDRKEALNYFSQIQDKPIVVKPKSTNFGLGISIFKTSANLASYEKAIDIAFTEDSAILVEEYIEGTEYRFFVLEGDCIAVLLRVAANVVGDGIHTISQLVKLKNQNPLRGYDHRSPLEVIELGEVEQLMLEQQGYTVNSIPPEGTKIELRRNSNISTGGDSIDVTNTMDPTYKQLAAEMAEAMGAWVCGVDLIIPNATQAYSKDKKNATCIELNFNPLMYMHTYCQEGPGQSITPRILAKLFPEL

SEQ ID NO: 6 - DNA, 2250 bp, synthetic construct
  source 1..2250: mol_type = other DNA
atgatcattgacagattattacaaagatctcactctcatttgccaatcttgcaagctactttcggtttggaaagagaatccttgcgtatccaccaaccaacccaaagagttgcccaaacccctcacccaaagaccttgggttctagaaactaccacccatacattcaaactgactactctgaaccacaactcgagctaatcacaccaattgccaaggattctcaagaagcgatccgtttcttgaaggccatttctgatgtcgccggtagatccattaaccatgatgaatatctatggccattgtccatgccacctaaggtccgtgaagaagacatacaaattgctcaactggaagatgctttcgaatacgattacagaaagtacttagaaaaaacttacggcaaattgatccaatccatctctggtatccactacaacttgggcttgggtcaagaattattgacttctttgtttgaattgtctcaagctgataacgctattgatttccaaaaccaactttacatgaagttgagtcaaaattttttgagatacagatggttgttgacctatttgtacggtgcttccccagttgctgaagaagacttcttagaccaaaagctcaacaacccagtcagatcgcttcgtaattcacacctaggttacgtcaaccacaaggacattagaatttcttacacttccttgaaagactacgttaatgacttagaaaacgctgtcaagagtggtcaattgatcgccgaaaaagaattctactccccagtcagattgcgtggttccaaggcttgtagaaactatttggaaaagggtattacttacttggagttcagaacttttgatttgaacccattctccccaattggtatcactcaagaaaccgtcgacaccgttcacttgttcttgttagccctattatggattgactcttcttctcacattgatcaagacatcaaggaagccaaccggttgaacgatctaatcgctttgtcccatccactcgaaaaattgccaaaccaagccccagtctctgacttagtcgatgccatgcaatctgttatccaacatttcaacttgtccccttactaccaagacttgttggaatctgtgaagagacaaattcaatctccagaattgactgtagctggtcaattgttagaaatgattgaaggtttgtctttggaaactttcggtcaaagacaaggtcaaatttaccacgactacgcttgggaagctccatacgctttgaagggttacgaaaccatggaattgtccactcaattgttgttgttcgacgttatccaaaagggtgtcaacttcgaagtcttggacgaacaagaccaattcttaaagttgtggcacaactctcacatcgaatacgttaagaacggtaacatgacttctaaggacaactatattgttccattggctatggctaacaaggttgtgactaagaagattttggatgaaaagcacttcccaaccccattcggtgacgaattcaccgacagaaaggaagctttgaactacttctctcaaatccaagataagccaattgtcgttaagccaaagtctactaacttcggtttgggtatttctattttcaagacttctgctaacttggcctcctacgaaaaggcaattgatatcgctttcactgaagattccgctatcctggtcgaagaatacatcgaaggtaccgaatacagattctttgtcttggaaggtgactgtattgctgttttgttgagagttgctgctaacgttgttggtgatggtatccacactatttcacagttggttaaactgaagaaccaaaacccgttgagaggttacgaccacagatctccattggaagttattgagttgggtgaagttgaacaattgatgttggaacaacaaggctatactgttaattcaatcccaccagaaggtactaagatcgaactaagaagaaactccaacatctctactggtggtgatagtattgatgttaccaacaccatggacccaacttacaagcaactagctgctgaaatggctgaagctatgggtgcttgggtttgcggtgtcgacttgatcatcccaaacgccacccaagcttactctaaggacaagaagaatgctacctgtatcgaattgaactttaacccattgatgtacatgcacacctactgtcaagaaggtccaggtcaatccatcaccccaagaatcttggctaaacttttcccagaatta